(19) United States
(12) Patent Application Publication
(10) Pub. No.: US 2012/0009590 A1
(43) Pub. Date: Jan. 12, 2012

(54) DEVICE AND NON-TOXIC COMPOSITIONS AND METHODS FOR PERMEABILIZATION OF DROSOPHILA EMBRYOS

(75) Inventor: Matthew D. Rand, Richmond, VT (US)

(73) Assignee: The University of Vermont and State Agriculture College, Burlington, VT (US)

(21) Appl. No.: 13/144,058

(22) PCT Filed: Jan. 11, 2010
(86) PCT No.: PCT/US2010/020626

§ 371 Date: Sep. 13, 2011

Related U.S. Application Data
(60) Provisional application No. 61/143,582, filed on Jan. 9, 2009.

(51) Int. Cl. C12Q 1/02; C12Q 1/18; C12M 3/00; C12N 5/07; G01N 33/566

(52) U.S. Cl. 435/7.1; 435/348; 435/29; 435/303.1; 435/32

(57) ABSTRACT

The invention provides compositions and methods for permeabilizing insect embryos by removing the waxy layer of the shell using a solution containing a non-toxic cyclic terpene and a non-toxic surfactant, preferably a non-ionic surfactant. The invention further provides kits to practice the method of the invention. The invention also provides methods for toxicology and other high throughput screening method including the compositions and methods of embryo permeabilization provided herein.

### CROSS REFERENCE TO RELATED APPLICATIONS

This application claims priority to U.S. Provisional Patent Application Ser. No. 61/143,582, filing date Jan. 9, 2009 which is incorporated herein by reference in its entirety.

### STATEMENT OF RIGHTS TO INVENTIONS MADE UNDER FEDERALLY SPONSORED RESEARCH

This work was supported in part by NIH, NIEHS R01ES015550. The government has certain rights in the invention.

### BACKGROUND OF THE INVENTION

Understanding toxicological mechanisms is ultimately a challenge of deciphering which genes and gene products in a developing or mature organism are targeted for disruption by chemicals encountered in the environment of the organism. The paucity of toxicological data for the more than 80,000 chemicals in commercial use today, and the approximately 2,000 new chemicals introduced each year make development of informative, sensitive and rapid assays to screen for toxicity paramount.

The large number of chemicals to be tested in combination with the ever increasing cost of maintaining even small animals (e.g., rats, mice) in animal facilities, as well as the relatively long life span of the animals, about 2-3 years, makes their use for toxicological screening time consuming and cost-prohibitive.

Understanding the susceptibility of the developing fetus to toxic environmental insult remains a primary concern in human health management. Assaying toxic mechanisms in embryos of mammalian models is, again, costly, time consuming and overshadowed with ethical ramifications.

### SUMMARY OF THE INVENTION

The invention provides non-toxic compositions and methods for permeabilization of membrane barriers of the Drosophila embryo shell to permit access of small molecules to embryonic tissues.

The invention provides methods of permeabilizing an insect embryo having a chorion layer and a waxy layer by sequentially removing a chorion layer from at least a portion of the insect embryo surface, preferably the entire insect embryo surface; and contacting the surface of the embryo with the chorion layer removed with a working dilution of an embryo permeabilization solution (EPS) comprising a non-toxic, monocyclic terpene and a non-toxic surfactant, whereby the insect embryo is permeabilized. In an embodiment, the insect embryo is an embryo from the order Diptera, especially the genuses Drosophila, Anopheles (mosquito), and Hymenopterans (especially ants and bees). In an embodiment the chorion layer is removed by contacting the embryo with a solution of sodium hypochlorite (NaClO) or manual dissection (e.g., using tape). In an embodiment the monocyclic terpene is a naturally derive mono-cyclic terpene such as limonene, particularly D-limonene, carvone, and cineole. In an embodiment, the surfactant is a non-ionic surfactant. In an embodiment, 2, 3, 4, 5, 6, 7, 8, 9, or 10 surfactants are used in combination with each other in EPS. In an embodiment the surfactants is an ethoxylated alcohol (C₉₋₁₁) and cocoanut diethanolimine (DEA).

The invention further provides a method for contacting the embryo with an agent suspected of comprising a biological activity after permeabilization of the embryo with EPS. In an embodiment, the agent has a molecular weight of 1500 Da or less, e.g., 1500 Da, 1400 Da, 1300 Da, 1200 Da, 1100 Da, 1000 Da, 900 Da, 800 Da, 700 Da, 600 Da, 500 Da, 400 Da, 300 Da, 200 Da, 100 Da, 50 Da, or any range of values bracket by the series of values. In an embodiment, the biological activity of the agent is a toxin activity. The methods of the invention can further include observing the embryo contacted with the agent as compared to an embryo not contacted with the agent, wherein a change in the embryo contacted with the agent indicates that the agent comprises biological activity. For example, the agent can result in an alteration of transcription from a promoter. The alteration in transcription from a promoter can be detected, for example, using a reporter gene or by PCR. The invention provides methods for contacting the embryo with more than one concentration of the agent. The invention provides methods for contacting the embryos with more than one agent (e.g., a potentially protective agent and a toxic agent). In an embodiment, the agent is soluble in aqueous solution.

The invention further provides a method for culturing a permeabilized insect embryo by transferring the EPS treated embryo into culture medium and maintaining the embryo in culture medium positioned such that at least a portion of the embryo surface is not submerged in the growth media to allow for gas exchange. For example, the portion of the embryo surface for gas exchange is about 10%-60%, 15%-50%, 20%-40%, or 20%-30% of the surface of the embryo. After transfer of the permeabilized embryos to culture media, at least 20%, 30%, 40%, 50%, 60%, 70%, 80%, or 90% of a population of embryos are viable for at least 3, hours, 6, hours, 9 hours, 12 hours, 18 hours, 24 hours, 36 hours, 48 hours, 72 hours, 96 hours, 5, days, or 6 days after transfer of the population of embryos to growth media when maintained at a temperature within the range from 16° C.-29° C. Viability can be tested by any of a number of methods including, but not limited to monitoring development and/or organogenesis. For example, after permeabilization and transfer into culture media, at least 20%, 30%, 40%, 50%, 60%, 70%, 80%, or 90% of a population of embryos will reach a developmental landmark, e.g., through 1, 2, 3, 4, 5, 6, 7, 8, 9, 10, 11, or 12 further developmental stages, to develop to the larval stage. Alternatively shorter time points can be monitored, e.g. mitotic events in the first 1.5, 3, 4, 5, 6, 7, 8, 10, 12 hours. The stage to which an embryo will develop will depend, in part, on the stage at which an embryo was permeabilized. For example, after permeabilization at Stage 4 and transfer into culture media, at least 20%, 30%, 40%, 50%, 60%, 70%, 80%, or 90% of a population of embryos will reach Stage 8 of development.

The invention further provides a method for monitoring the embryo for permeabilization and/or viability using a detectable label, for example a fluorescent label, for example an intercalating fluorescent label. Alternatively a fluorescent label that can be metabolized by a viable embryo resulting in a shift in absorbance-emission of specific wavelengths of light. In a preferred embodiment, the permeabilized embryos can be contacted with the detectable label simultaneously with another agent to be tested for biological activity. In an alternate embodiment, a large population of embryos can be permeabilized and divided into discrete pools that can be tested for response to an agent that may have biological activity while another pool can be tested to determine percent viability and/or permeability.

The invention provides EPS having non-toxic cyclic terpene at a concentration of about 70%-90%, about 72%-88%, about 75%-85%, about 78%-83%, about 79%-81% of the volume of the EPS; and the total surfactant present at a concentration of about 10%-about 30%, about 12%-28%, about 15%-25%, about 17%-22%, about 19%-21%, about 20% of the volume of the EPS. The number of surfactants present is 1, 2, 3, 4, 5, 6, 7, 8, 9, 10. or more surfactants. The invention provides a working solution of EPS that is about a 1:1, 1:2, 1:5, 1:10, 1:20, 1:301:40, 1:50, 1:60, 1:70, 1:80, 1:90, or 1:100 dilution of EPS. The invention provides a working solution of EPS that includes about 0.1%-10%, about 0.5%-10%, about 1%-10%, about 2%-10%, about 0.1%-6%, about 0.5%-5%, about 0.5%-4%, about 1% to 4%, about 1% to 3%, about 0.1%-3%, about 0.5%-6% of total surfactant by volume and about 0.7%-18%, about 0.7%-15%, about 1%-15%, about 3%-12%, about 3%-10%, about 5%-15%, about 8%-12%, about 1%-45%, about 1%-30%, about 1%-20%, about 3%-45%, about 3%-30%, about 3%-20% non-toxic cyclic terpene by volume.

The invention provides EPS with one or more non-ionic surfactants including, but not limited to alkyl poly(ethylene oxide), ethoxylated coconut alcohol, alcohol ethoxylate (C₉₋₁₁), copolymers of poly(ethylene oxide) and poly(propylene oxide); alkyl polyglucosides; fatty alcohols; cocamide MEA, cocamide DEA; polysorbates, ethoxylated fatty alcohol ethers and lauryl ethers, ethoxylated alkyl phenols, octylphenoxy polyethoxy ethanol compounds, modified oxyethylated and/or oxypropylated straight-chain alcohols, polyethylene glycol monooleate compounds, polysorbate compounds, and phenolic fatty alcohol ethers.

The invention further provides a method whereby the surface of the embryo with the chorion layer removed is contacted with a working dilution of an embryo permeabilization solution (EPS) in an embryo incubation device including a sidewall enclosing a space having a top opening and a bottom opening; a mesh bottom covering the bottom opening; and a base for elevating the mesh bottom comprising at least two openings, wherein the embryo incubation device is placed in a chamber containing a working solution of EPS such that the EPS contacts the mesh bottom of the device while exposing a surface of the embryo for gas exchange. The embryo surface for gas exchange is preferably about 10%-60%, 15%-50%, 20%-40%, or 20%-30% of the surface of the embryo. The invention includes the assembly of multiple embryo incubation devices into an assembly (e.g., similar to a 96-well or 384-well plate). In such a format, each embryo incubation device need not include a separate base for elevating the bottom of the well from the surface of the chamber. A single base to elevate all of the wells in the assembly could be used. Such modifications are well within the ability of those of skill in the art. The invention includes methods wherein multiple embryo incubation devices are incubated in EPS simultaneously.

The invention provides an embryo incubation device including a sidewall enclosing a space comprising a top opening and a bottom opening; a mesh bottom covering the bottom opening; and a base for elevating the mesh bottom comprising at least two openings. The invention further provides an assembly of multiple embryo incubation devices into an assembly (e.g., similar to a 96-well or 384-well plate). In such a format, each embryo incubation device need not include a separate base for elevating the bottom of the well from the surface of the chamber. A single base to elevate all of the wells in the assembly could be used. Such modifications are well within the ability of those of skill in the art.

The invention provides EPS solutions, either concentrated or as a working dilution, such as those provided for use in the methods provided herein.

The invention provides kits including an EPS solution and instructions for use in appropriate packaging.

### DEFINITIONS

The term “agent” or “toxic agent” and the like are understood as a compound (e.g., small molecule, nucleic acid, protein, carbohydrate, etc.) that has or is suspected of having a biological activity, potentially biological activity as a toxin.

By “alteration” is meant a change (increase or decrease) in the expression levels or activity of a gene or polypeptide as detected by standard art known methods (e.g., ELISA, PCR, fluorescent reporter gene expression). As used herein, an alteration includes at least a 20% change in reporter gene activity, preferably at least a 35% change, preferably at least a 50% change, and preferably at least a 70%, preferably at least a 80%, preferably at least a 90%, preferably at least a 100%, or greater change in expression levels. Alteration as used herein can also refer to a change in frequency of a specific characteristic or condition in a population. For example, an alteration can result in a change in the frequency of a specific condition to a rate at least about 10%, at least about 20%, at least about 35%, at least about 50%, at least about 70%, at least about 80%, at least about 90%, at least about 100%, or more in the frequency of the specific condition.

By “analog” or “derivative” is meant a molecule that is not identical, but has analogous functional or structural features.

By “contacting a cell” is meant providing an agent to a cell in a manner that the agent can have an effect on the cell. For example, in culture, an agent can be added to culture media. In an animal, the agent can be administered by any parenteral (e.g., microinjection), enteral (e.g., by feeding), or topical route of administration that allows at least a portion of the agent to be delivered to the cell to be contacted. In certain embodiments, a cell of a Drosophila embryo is contacted after permeabilizing the embryo shell.

The term “detectable label” is understood as a chemical, chemical modification, binding agent, or other tag that can be readily observed, preferably in a quantitative manner, such as a fluorescent tag that has a specific wavelengths of absorption and emission to allow detection of the compound associated with the detectable label. Detectable labels include vital dyes for use with living cells wherein the dye has little or no effect on cell viability, growth, and function. Detectable labels also include dyes of various sizes detectable in either the visible or non-visible light range. Multiple detectable labels that can be visually distinguished can be used simultaneously.

By “detecting” or “detection” and the like is meant the process of performing the steps to determine if an analyte is present, or to identify an agent with a biological activity, e.g., a toxicity. The amount of analyte present may be none or below the level of detection of the method.

By “diagnosing” as used herein refers to a clinical or other assessment of the condition of a subject based on observation, testing, or circumstances for identifying a subject having a disease, disorder, or condition based on the presence of at least one sign or symptom of the disease, disorder, or condition. Typically, diagnosing using the method of the invention includes the observation of the subject for other signs or symptoms of the disease, disorder, or condition. As used herein, diagnosis can also include detection of a disease or condition, e.g., lack of normal development, lack of viability, of a Drosophilid.

The term “differential” or “differentially” generally refers to a statistically significant different level in the specified property or effect. Preferably, the difference is also functionally significant. Likewise, “differential effect” or “differentially active” in connection with a toxin refers to a difference in the level of the effect or activity that is distinguishable using relevant parameters and techniques for measuring the effect or activity being considered.

As used herein, “Drosophila” is understood as a member of the Dipteran order and the genus and species Drosophila melanogaster. However, the methods of the invention for permeabilization of embryos can be used with any type of embryo that has a waxy layer similar to that present in Drosophila, and common to many Dipterans (e.g., house fly (Musca domestica) and mosquito (Anopheles gambiae)), Lepidopterans (butterflies) and Hymenopterans (bees and wasps).

By “embryo permeabilization solution” or “EPS” is meant a combination of at least one non-toxic cyclic terpene, preferably a plant derived non-toxic cyclic terpene e.g. from eucalyptus (Cineole), or the citrus derived compound D-limonene, in combination with at least one non-toxic surfactant (e.g., 1, 2, 3, 4, 5, 6, 7, 8, 9, 01, or more) in sufficient quantity to allow the EPS solution to be miscible in water at a 1:1 to 1:100 dilution range wherein the EPS is a stable emulsion that remains a visibly homogeneous solution for at least about 12 hours, at least about 24 hours, at least about 48 hours, at least about a week after thoroughly mixing the solution. In an embodiment, the surfactant includes a combination of at least two surfactants, preferably non-ionic surfactants. In a preferred embodiment, the surfactant is about 10%-about 30% (v/v) of the EPS, about 12%-about 28% (v/v) of the EPS, about 15%-about 25% (v/v) of the EPS, about 17%-about 22% (v/v) of the EPS, about 19%-about 21% (v/v) of the EPS, about 20% (v/v) of the EPS, with the remaining volume preferably provided by the non-toxic cyclic terpene (e.g., about 70%-about 90%, about 72%-about 88%, about 75%-about 85%, about 78%-about 83%, about 79%-about 81%), however other agents may be present provided that the agents do not prevent the formation of a stable emulsion between the cyclic terpene and the surfactants. In an embodiment, the ratio of the first surfactant to the second surfactant is about 1:50, about 1:25, about 1:20, about 1:15, about 1:10, about 1:5, about 1:3, about 1:2, about 1:1, about 2:1, about 3:1, about 5:1, about 10:1, about 15:1, about 20:1, about 25:1, or about 50:1.

Typically EPS is used at a working concentration or as a working solution of about a 1:5 to about a 1:100 (v/v) dilution in an aqueous solution such as water, however, EPS can be diluted at other dilutions e.g., a 1:1. 1:2, 1:5, 1:10, 1:20, 1:30, 1:40, 1:50, 1:60, 1:70, 1:80, 1:90, or 1:100 dilution. Such a diluted solution of EPS can be referred to as a “working solution.” Therefore, a working solution of EPS would include about 0.1%-about 6% total surfactant and about 0.7% to about 18% cyclic terpene, however, other ranges are possible.

EPS diluted in aqueous solvent does not necessarily form a stable emulsion for an extended period, but should not visibly separate into aqueous and non-aqueous phases during the permeabilization of the embryos, e.g., within about 10 minutes, within about 15 minutes, within about 30 minutes, within about 45 minutes, within about 60 minutes. Diluted EPS may be a cloudy solution. A working solution of EPS can further include any of a number of components such as buffering agents, salts, and proteins that may be useful to maintain the integrity of the embryos.

As used herein, an agent “incompatible with life” is understood as an agent that results in a loss of viability of the organism. Loss of viability need not be immediate. For example, an agent can inhibit cell division or cell growth in the embryo, resulting in stasis followed by death. An agent can cause extensive damage to DNA, cell membranes, or proteins resulting in death of the embryo. Agents can act as toxins to the cell almost immediately by inhibiting essential cell processes.

As used herein, “isolated” or “purified” is understood as being removed from its usual environment or other components with which they are naturally associated. For example, an isolated cell can be removed from an animal and placed in a culture dish or another animal Isolated is not meant as being removed from all other cells. A group of cells can also be isolated (e.g., bone marrow cells, an organ) from one organism and placed in another organism or in culture. A polypeptide or nucleic acid is isolated when it is about 80% free, 85% free, 90% free, 95% free from other cellular material typically associated with the nucleic acid or polypeptide (e.g., material in a cell in which the nucleic acid or peptide is expressed), or substantially free of chemical precursors or other chemicals when chemically synthesized. An “isolated polypeptide” or “isolated polynucleotide” is, therefore, a substantially purified polypeptide or polynucleotide, respectively. As used herein, a “homogenously purified” polypeptide or nucleic acid is about 90%, 95%, 98%, or 99% pure and removed from biological or synthetic contamination from the synthesis of the polypeptide or nucleic acid. A homogeneously purified polypeptide or nucleic acid can be present in a buffer or other carrier such as a pharmaceutically acceptable carrier.

“Limonene” is a cyclic terpene that occurs naturally in citrus fruits, particularly in the peel. Limonene has the following structure:

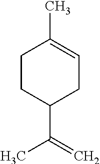

As used herein, a “normal cell” is a cell that is derived from tissue that does not include any known mutations or disruptions that predispose the cell to a particular disease or disorder. A normal cell can also be an untreated cell, i.e. a cell not contacted with a toxin or an agent suspected of being a toxin, but possibly contacted with a carrier substance not expected to have any activity. Such a cell is also known as a control cell.

As used herein, “obtaining” is understood as purchase, procure, manufacture, or otherwise come into possession of the desired material.

“Permeabilize” or “permeable to agents” and the like as used herein refers to an increase in the ability of agents or compounds to pass more readily through the shell of the embryo to the cells within the embryo. In an embodiment, the embryo is permeabilized to agents that can be dissolved in pharmaceutically acceptable carriers, preferably aqueous solutions that do not have detrimental effects on the embryos. In an embodiment, the embryos are permeable to compounds that would not normally pass through the shell of the embryo. In an embodiment, the compounds have a molecular weight of at least 100 Da, 500 Da, 1000 Da, 1250 Da, 1500 Da, 1750 Da, 2000 Da, 2500 Da, 3000 Da, 4000 Da, or 5000 Da. Not all compounds of a predetermined size need to be able to pass through the shell of the embryo. For example, it is understood that a compound that has poor solubility in the solution in which the permeabilized embryo is contained will not likely pass through the shell of the embryo. In an embodiment, the compound is soluble in water. In an embodiment, the compound is soluble in a 1:10 dilution of EPS in water.

As used herein, the term “polymerase chain reaction” (PCR) refers to the methods of U.S. Pat. Nos. 4,683,195, 4,683,202, and 4,965,188, all of which are hereby incorporated by reference, directed to methods for increasing the concentration of a segment of a target sequence in a mixture of genomic DNA, or messenger RNA, without cloning or purification. As used herein, the terms “PCR product” and “amplification product” refer to the resultant mixture of compounds after two or more cycles of the PCR steps of denaturation, annealing and extension are complete. These terms encompass the case where there has been amplification of one or more segments of one or more target sequences.

The polymerase chain reaction (PCR) is a widely known method for amplifying nucleic acids. Of the PCR techniques, RT-PCR (Reverse Transcription-PCR), competitive RT-PCR and the like are used for detecting and quantifying a trace amount of mRNA, and show their effectiveness. A real-time quantitative detection technique using PCR has been established (TaqMan® PCR, Genome Res., 6 (10), 986 (1996), ABI PRISM™ Sequence Detection System, Applied Biosystems, incorporated herein by reference). Continuous fluorescence monitoring of rapid cycle DNA amplification. Wittwer C T, Herrmann M G, Moss A A, Rasmussen R P. Biotechniques. 1997 January; 22(1):130-1, 134-8. PMID: 8994660 (The SYBR® Green dye is sold pursuant to a limited license from Molecular Probes, Inc. under U.S. Pat. Nos. 5,436,134 and 5,658,751 and corresponding foreign patents and patent applications.)

The terms “polypeptide,” “peptide” and “protein” are used interchangeably herein to refer to a polymer of at least three amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

By “population” is meant a group of organisms, i.e., at least two. The number of organisms in a population is preferably sufficient to allow for determination of the effects of one or more compounds on the organism. For example, in methods of the invention, populations of embryos can be placed in individual wells of 96-well or 348-well plates for permeabilization and/or contacting the embryos with a compound of interest for toxicology testing. The rate of death or dysfunction in the population contacted with the potential toxin can be compared to a population not contacted with the toxin, or contacted with a potentially protective agent in conjunction with the toxin. The number of embryos in each of the populations need not be identical. In some embodiments, a larger population of embryos can be contacted with a first agent, and subsequently divided and contacted with a second agent. The number of embryos in a population can be, for example about 5 to about 500, or more. For example, the number of embryos in a population can be about 5-15 embryos, about 10-50 embryos, about 10-100 embryos, about 50-100 embryos, about 25-250 embryos, about 100-500 embryos, or about 200-500 embryos. Preferably, the population is of a sufficiently large size to provide statistically significant results.

“Providing,” refers to obtaining, by for example, buying or making the, e.g., polypeptide, drug, polynucleotide, probe, and the like, including libraries of such compounds or libraries of combinations of types of compounds. The material provided may be made by any known or later developed biochemical or other technique. For example, compounds may be derived from natural sources, be chemically synthesized by directed or combinatorial methods, or a collection of known compounds (e.g., compounds approved for therapeutic use in humans). Materials can be obtained from sites including unknown compounds including sites containing or suspected of containing environmental toxins.

The term “recombinant DNA molecule” as used herein refers to a DNA molecule, which is comprised of segments of DNA joined together by means of molecular biological techniques.

As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “reporter gene” which is typically used in the context of a “reporter construct” which is understood as a coding sequence for a polypeptide that is operably linked to a promoter sequence, typically an inducible promoter. Alternatively, the reporter gene can be under the control of a constitutive promoter that can be turned off in response to a specific stimulus. The coding sequence for a polypeptide encodes a polypeptide that can be readily detected, preferably in a quantitative manner, such as alkaline phosphatase, beta-galactosidase, luciferase, green fluorescent protein, etc. Methods to detect the presence of such proteins is well known in the art and can be performed using any of a number of commercially available kits and automated readers. The selection of a reporter gene is a matter of choice.

As used herein, “selecting” is understood as identifying one or more members of a group.

By “small molecule” is meant a compound having a molecular weight of no more than about 1500 Daltons, 1000 Daltons, 750 Daltons, 500 Daltons. A small molecule, as used herein, can include a nucleic acid or polypeptide. Small molecule, as used herein, can exclude nucleic acid or polypeptide.

By “solution” or “solvent” is meant a liquid that is free-flowing at room temperature (about 20-22° C.). Solutions and solvents can be organic or inorganic.

The term “subject” includes organisms which are capable of suffering from a disease of interest that could be susceptible to exposure to a toxin or a compound suspected of being a toxin, such as human and non-human animals, including fetuses or embryos. The term “non-human animals” of the invention includes all vertebrates, e.g., mammals, e.g., sheep, dog, cow, and primates including non-human primates; e.g., rodents, e.g., mice, and non-mammals, e.g., chickens, amphibians, reptiles, insects etc. A human subject can be referred to as a patient.

“Surfactant” is a blend of “surface acting agent”. Surfactants are usually organic compounds that are amphiphilic, meaning they contain both hydrophobic groups and hydrophilic groups. Therefore, they are soluble in both organic solvents and water. Surfactants can be used to solubilize compounds that are only slightly or not completely soluble in either an aqueous or organic solvent. Surfactants reduce the surface tension of water by adsorbing at the liquid-gas interface. They also reduce the interfacial tension between oil and water by adsorbing at the liquid-liquid interface. Surfactants can be chemically synthesized or naturally occurring, or chemically modified naturally occurring compounds. Examples of such detergents include those specified on pages 295-298 of McCutcheon's Emulsifiers & Detergents, North American edition (2008), published by the McCutcheon Division of MC Publishing Co., (USA), the entire disclosure of which is incorporated herein by reference. Detergents are typically surfactants.

Surfactants are commonly divided into groups based on their charge, for example, anionic (e.g., based on sulfate, sulfonate or carboxylate anions), cationic (based on quaternary ammonium cations), zwitterionic (amphoteric), and non-ionic (e.g., alkyl poly(ethylene oxide) such as ethoxylated castor oil (CAS# 61791-12-6) and ethoxylated coconut alcohol (CAS# 68131-39-5); copolymers of poly(ethylene oxide) and poly(propylene oxide); alkyl polyglucosides; fatty alcohols; cocamide MEA, cocamide DEA; and polysorbates). Surfactants can also be divided into groups based on their molecular weight, their critical micelle concentration (CMC) and micelle molecular weight (mMW).

Non-ionic surfactants include, but are not limited to, ethoxylated fatty alcohol ethers and lauryl ethers, ethoxylated alkyl phenols, octylphenoxy polyethoxy ethanol compounds, modified oxyethylated and/or oxypropylated straight-chain alcohols, polyethylene glycol monooleate compounds, polysorbate compounds, and phenolic fatty alcohol ethers. More particularly preferred are Tween® 20, from ICI Americas Inc., Wilmington, Del., which is a polyoxyethylated (20) sorbitan monolaurate, and Iconol™ NP-40, from BASF® Wyandotte Corp. Parsippany, N.J., which is an ethoxylated alkyl phenol (nonyl). Non-ionic detergents include alcohol ethoxylates, for example C₉₋₁₁ chain length, C₈₋₁₂ chain length, C₉₋₁₂ chain length and diethanolimide or diethanolamine, particularly derived from natural sources such as cocoanut (e.g., commercially available from the Steppan Company, Northfield, Ill.).

As used herein, “susceptible to” or “prone to” a specific disease or condition and the like refers to an individual who based on genetic, environmental, health, and/or other risk factors is more likely to develop a disease or condition than the general population. For example, a large number of strains of Drosophila are available that have a number of well characterized phenotypes associated with specific genotypes that result in specific developmental and/or physiological characteristics, including chemical sensitivities, morphological abnormalities at the cellular and organ level, or behavioral characteristics.

A “terpene” as used herein is an organic compound including at least one isoprene unit, shown below. As used herein, a cyclic terpene includes a saturated or unsaturated 6 member carbon ring in addition to an isoprene substituent.

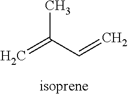

“Toxicology” as used herein is the study of the relationship between dose and its effects on the exposed organism. The chief criterion regarding the toxicity of a chemical is the dose, i.e. the amount of exposure to the substance.

The term “toxin” as used herein is understood as an agent that is detrimental to the health or well-being of a subject, including agents that are detrimental to the development of a subject. A toxin need not be fatal or be detrimental at all doses or after a single dose (e.g., water can be a toxin at a sufficiently high dose). As used herein, a toxin can be identified using a dose-response curve or other method to determine at what level the compound becomes a toxin or demonstrates detrimental effects on the organism tested. Therefore, as used herein, the invention provides a non-toxic composition for permeabilization of embryos in which the concentration of the components of the solution will determine if the solution is toxic or non-toxic. As used herein, exposure of a population of embryos to a non-toxic compound or solution preferably results in death or cessation of development of less than about 15%, less than about 10%, less than about 8%, less than about 5%, less than about 4%, less than about 3%, less than about 2%, less than about 1% under the time and temperature conditions being used to test a compound for toxicity.

Toxicity of a compound can also be determined relative to a control. For example, treatment of embryos with a working solution of EPS may prevent the embryos, especially certain strains of embryos, from undergoing various steps of development optionally to emerge as larva. In such a case, toxicity of agents to be tested is compared to a control to determine if the compound hastens death or cessation of development. For example, if the compound results in death or cessation of development by at least about 1 hour, at least about 2 hours, at least about 4 hours, at least about 8 hours faster than an embryo not treated with the agent being tested for toxicity. In an embodiment, the embryos are incubated at about 20° C., in an alternative embodiment, the embryos are incubated at about 25° C. In an embodiment, the embryos are incubated at any temperature from about 18° C. to about 37° C. Therefore, as used herein, exposure of a population of embryos to a non-toxic compound or solution preferably hastens death of less than about 15%, less than about 10%, less than about 8%, less than about 5%, less than about 4%, less than about 3%, less than about 2%, less than about 1% of the embryos under the time and temperature conditions being used to test a compound for toxicity. Toxicity in embryos can readily be tested empirically by direct observation (with microscopy) of cessation in development over an 8 hour window of incubation.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. All values provided herein are understood to be modified by the term about.

Dilutions provided are understood as the parts of a specific component into the total number of parts in the solution. For example, a 1:10 dilution of composition X would be 1 ml of composition X+9 ml of diluent (e.g., water, buffer, growth media)=10 total parts.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. For example, any single point mutation or alteration of the wild-type sequence can be combined with any other mutation or alteration of the wild-type sequence. Any Drosophila strain can be crossed with any other Drosophila strain.

Any compositions or methods provided herein can be combined with one or more of any of the other compositions and methods provided herein.

Unless otherwise stated, all ratios of solutions or percent solutions are volume/volume.

### BRIEF DESCRIPTION OF THE DRAWINGS

FIG. 1 shows a device for contacting insect embryos with solutions sequentially which can be used in the methods of the invention. Embryos (E) are suspended by a nitex screen in a basket (B). The basket is placed in a reservoir (R) which contain medium suitable for embryo development and agent delivery.

FIG. 2A-B. (A) shows a schematic of an embodiment of the method of the invention including the steps of embryo collection, dechorionation and permeabilization, staining, optional exposure to one or more agents and/or culture conditions, and imaging of embryos. (B) shows modes in which the Drosophila model can be used in toxicological assays. Components of the scheme are the variety of chemical toxicants to be investigated; the mode of delivery to the organism; the developmental stage of the fly; and the endpoints to be measured in determining biological/toxicological effects. Methods herein are applicable to embryonic manipulations.

FIG. 3A-E. (A) shows permeabilization of Drosophila embryos. Non-EPS treated embryos immersed in cresyl violet (0.05% in PBS) show no incorporation of the dye. (B) shows EPS-treated embryos immersed in cresyl violet, (C) fast green (0.05% in PBS), or (D) rhodamine B which show dye uptake in 30 minutes.

FIG. 4A-B (A) shows a schematic of the Drosophila egg shell layers. From the inside out are the vitelline membrane (VM), waxy layer (WL), inner-, endo- and exochorionic layers, respectively (ICL, EN, EX). (Margaritis et al, J Cell Sci 43:1-135, 1980) (B) shows scanning EM images of the control treated embryos (top) and EPS treated embryos (bottom) having a smooth and homogeneous surface, indicating removal of the waxy plaque layer and exposure of the vitelline membrane proper.

FIG. 5 shows delivery of Syto11® nucleic acid stain to embryos. (A-C) Non-EPS treated embryos immersed Syto11® (5 μM in PBS) show exclusion of the stain. EPS-treated embryos immersed in Syto11® show staining of cytosol and nuclei at several cell layers in (E) stage 6 (˜3 hour old) and (F) stage 11 (˜8 hour old) embryos. Images captured with (A,D) epiflourescence and (B,C,E,F) confocal microscopy.

FIG. 6A-F show the permeability of EPS treated embryos using a panel of fluorescent dyes imaged using confocal microscopy. (A, C, and D) show Rhodamine B staining in the cytosol of pre-blastoderm embryos as well as in the first cells formed at the blastoderm stage, but excluded from the nucleus (D). (B and C) show Calcein AM (MW=944 Da) staining in the periphery of a blastoderm embryo. (E and F) show Syto 11 strongly label the condensed chromosomes of pre-mitotic figures of an early embryo (undergoing nuclear division 13) (E) and at the periphery of an embryo undergoing gastrulation (F).

FIG. 7A-B show developmental outcomes of embryos based on their degree of rhodamine B uptake. Embryos were sorted by eye for staining intensity and organized into rows of lowest dye uptake (top row) to highest dye uptake (bottom row). Embryos were imaged by (A) bright field to assess development, and by fluorescence microscopy (B) to assess rhodamine uptake in the red channel, and (C) to assess metabolism of rhodamine in the green channel.

FIG. 8 shows an anterior to posterior gradient of Rhodamine B distribution in embryos over a time course with images taken at 10, 30, and 90 minutes as indicated. The cephalic furrow is indicated by cf in the 90 minute image and the pole cells are indicated by the open arrow at the 30 and 90 minute time points.

FIG. 9A-B. (A) shows stages of embryo development and neurogenesis. Anterior is left and dorsal is up. Neuroblasts (NBs, light purple) form at stage 8-11. Ventral nerve cord neurons (VNC, Purple) arise from NBs from stage 11-15. The VNC retracts in stages 12-17. (midgut/hindgut: red/blue). (Adapted from V. Hartenstein, Atlas of Drosophila Development, 1993). (B) shows embryos permeabilized with a standard treatment of 1:10 diluted EPSA for 30-60 seconds at stages of development up to stage 13 and stained with rhodamine B. Bright field (left column) and fluorescent images (right column) are shown.

FIG. 10A-B shows (A) permeabilized and (B) non-permeabilized embryos treated with rhodamine B dye and imaged at indicated times (minutes) of development after rhodamine B staining. Within each group, the left column shows fluorescence in the green channel and the right column shows fluorescence in the red channel.

FIG. 11A-E. shows monitoring development under fluorescence time-lapse imaging with or without exposure to the toxin cycloheximide (CHX). (A) shows the highly dynamic pattern of blue fluorescence emitted from the auto-fluorescence of yolk proteins in the embryo after permeabilization, but not treated with any additional agents at hours after laying indicated. Images B and D are bright field and images C and E are fluorescence images of embryos (B and C) not treated or (D and E) treated with cycloheximide.

FIG. 12A-B shows exemplary embryos that (A) were not or (B) were permeabilized prior to treatment with cycloheximide and staining with rhodamine B to assess the efficacy of permeabilization. Embryos were imaged by (A and B) differential interference contrast or (A′ and B′) fluorescence microscopy and scored for normal or abnormal cephalic furrow formation and germband elongation. Results are shown in Table 1. n≧180 per group.

FIGS. 13 A-E. (A and B) show the dynamic distribution of Dorsal-GFP in and out of the ventral nuclei of an EPS treated pre-blastoderm embryos in response to various conditions in (A) embryos imaged in red and green channels simultaneously or in (B) red (top row) and green channels (bottom row) individually. (C-E) show the pattern of nuclear divisions and Dorsal distribution in preblastoderm embryos in response to cyclohexamide treatment (C) when the syncytial nuclei first appear at the embryo surface, (D) at slightly later stages when irregular nuclear Dorsal accumulation is observed, and (E) the formation of various sized nuclei at the 13-14 nuclear division stage

FIGS. 14A-D show the patterning of (A) engrailed-GFP in non-permeabilized (top row) and permeabilized (bottom row) embryos in response to (B) cytochalasin D, (C) methylmercury, and (D) cyclophosphamide.

FIG. 15A-B shows automated analysis of GFP expressing embryos. (A) shows expression of neural-specific GFP in embryos. Embryos imaged by epiflourescence at the indicated stage of development are shown. A neurogenic embryo displays an expanded region of GFP expression. (B) shows detection of neural-specific-GFP signal in embryo samples using a plate reader format. Fluorescence signal (arbitrary units, background corrected) of increasing numbers of embryos positioned in wells of 96-well plate.

FIG. 16 shows a scheme for a high throughput screening platform of embryo assay. Embryos collected from a population cage are dechorionated and permeabilized in nitex baskets (see FIG. 1). Recommended instrumentation is pictured for multiwell distribution of embryos (COPAS SELECT®, Union Biometrica) and image capture and analysis (BD® Bioimager, BD Biosciences).

### DETAILED DESCRIPTION OF THE INVENTION

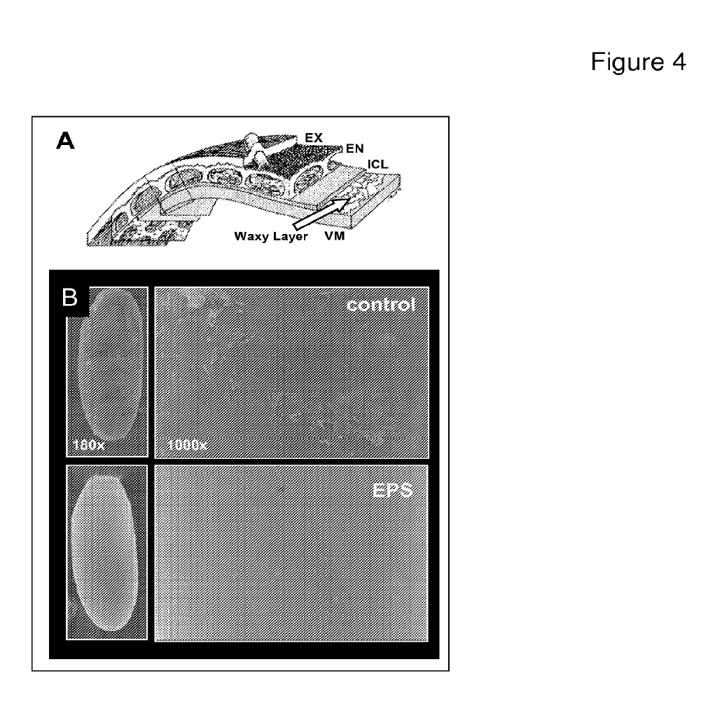

Our objective was to render the fly embryo permeable to small molecules. The eggshell is comprised of three chorion layers, a waxy layer and the vitelline membrane (see FIG. 4A). Chorion layers are easily removed with sodium hypochlorite treatment with no adverse effects on the embryo (Strecker, 1994; Hill, 1945). Permeability is almost entirely restricted by the waxy layer and thus we sought to optimize methods to remove this layer. D-Limonene (referred to as limonene hereafter) is a monoterpene oil that is the predominant constituent of oil in the peel of oranges. Limonene has proven to be an exceptionally effective substitute for petroleum based solvents for removal of oily and waxy substances and has found numerous applications in industrial processes and in common household “citrus” cleaners. Limonene is a FDA registered GRAS (generally regarded as safe) substance approved for use as a food additive (Opdyke, 1975). Limonene exhibits minimal toxicity in a number of biological systems, however it does illicit toxic responses at very high doses (IPCS, 2009; NTP, 1990). We demonstrate herein that a limonene-based solvent is a desirable substitute for the conventional heptane or octane applications used to remove the waxy layer. Furthermore, in the presence of suitable surfactants limonene is miscible with water, which eliminates the need to transition embryos in and out an organic phase to achieve waxy layer removal.

We provide a method to render the Drosophila embryo eggshell permeable to small molecules while maintaining conditions suitable for continued development of the embryo in vitro. This method overcomes a longstanding barrier for routine application of small biologically active molecules to the developing fly embryo and thus opens a door to pharmacological approaches to developmental studies in this premier model organism. In addition, this method reveals the potential of the Drosophila embryo for advancing toxicological studies aimed at characterizing large libraries of compounds. A number of the technical challenges we encountered in developing this method are worthy of discussion since they shed light on the biology and mechanics of the system.

To date successful methods of permeablizing embryos have utilized the organic solvents octane or heptane (Limbourg, 1973; Mazur, 1992; Strecker, 1995). However, two features of this approach have presented difficulties and have limited widespread use of permeabilizaion as a technique. These difficulties are: 1) the need to transition embryos out of and back into an aqueous medium results in extreme sensitivity to desiccation and 2) the degree of permeabilization is difficult to control due to the brief exposure necessary with these strong solvents. Our method improves upon both of these limitations of using organic solvents. First, the ability to use EPS as an aqueous emulsion avoids difficulties of desiccation by eliminating transitions between aqueous to organic phases. Second, the ability to vary EPS concentration enables more precise control of permeabilization of the embryo shell. The outcome of these improvements is that a higher yield of permeabilized viable embryos can be obtained in batch preparations.

Despite these improvements, our findings reinforce the previous observations of Mazur et al. (1992) that embryo viability is very sensitive to permeabilization. This observation has pointed to the importance of defining the threshold of permeabilization that adversely effects development. We have advanced the ability to determine embryo permeability by using the fluorescent properties of rhodamine B. We establish that viability is highest in the window where rhodamine B uptake is easily detected by fourescence but hardly detected under brightfield illumination. Importantly, we see that the qualitative trait of the “gradient” of rhodamine B dye uptake in the first 15-20 minutes after dye exposure serves as a reliable indicator of an optimally permeabilized embryo. Furthermore, we identify the characteristic that permeability decreases with age of the embryo, which can be accommodated for by longer incubations in EPS. Thus, for general laboratory practice, familiarity with the present protocol will give the user enough experience to identify optimally permeabilized embryos of any age using standard fluorescence microscopy. The user can manually selected and set up embryos for drug treatments in baskets or the slide chamber described here.

Our method has potential applications in high throughput analysis of small molecules. The small size of the embryo (150×500 μm) permits culturing of >50 embryos in the well of a 96 well plate, and the possibility of culturing in higher density format (e.g. 384 well). Commercial instruments are available that can sort based on fluorescent properties (intensity and distribution) and distribute embryos into multiwell formats (e.g. the COPAS SELECT instrument by Union Biometrica, Holliston, Mass.). In addition, automated confocal image capture instrumentation is commercially available that can acquire images from a 96-well format (e.g. the BD Pathway 855 from BD Biosciences). Software associated with these instruments is capable of quantifying fluorescence intensity and distribution across the dimensions of the embryo. We predict that application of the this method to the numerous GFP reporter transgenic fly lines currently available holds the promise of being able to determine activity of a large number of compounds on a wide variety of developmental endpoints in the embryo. One relevant embodiment of this technology is a screening platform using a collection of fly strains that report for each of the major intercellular signaling pathways (e.g. Notch, Wnt, hedgehog). Systematic evaluation of response of these embryos to various classes and concentrations of drugs/toxins could yield highly informative mechanistic data to advance the understanding of the bioactivity of compounds of interest as well as identify tools to investigate individual pathways.

We have shown through proof of principle experiments that EPS is highly effective in permeabilizing the Drosophila eggshell. The invention allows for the introduction of a wide array of small molecules to the developing embryo. A few parameters that will be resolved with further evaluation will be: 1) limitations to the ability to delivery a variety of classes of compounds (e.g. membrane permeable vs. impermeable), 2) limitation on the size of compounds that can be delivered, 3) compatibility of permeabilized embryo culture with carrier solvents (e.g. DMSO). Nonetheless, under conditions reported here the method holds promise for advancing pharamacological approaches to investigating developmental mechanisms of the fly embryo. One aspect of note revealed in this study is the increased resistance to permeabilization with developmental age. This observation points to a “hardening” mechanism in the eggshell that occurs post ovoposition. A post-ovoposition hardening mechanism as has been reported in mosquito embryos but is less clear in Drosophila. Use of EPS could prove useful in determining the contribution of the wax layer to this process. EPS wax layer removal should be equally effective in permabilizing eggshells of other Dipterans (e.g. mosquitoes) and insect species that present a wax layer composition in their eggshell structure, allowing for analysis of chemical effects and other analyses of such organisms.

The invention provides devices, non-toxic compositions, and methods for the permeabilization of Drosophila embryos, particularly the removal of the waxy layer of the Drosophila embryo shell, allowing for the passage of small molecules and other compounds not typically able to pass through the waxy layer into the embryo without the need for manipulating the embryos individually (e.g., as with microinjection). The invention also overcomes the potentially toxic side effects of the use of octane or other organic solvents that have been used previously to remove the waxy layer of the embryo. The use of an aqueous solution provides the further advantage of being a better solute for many pharmaceutical agents and toxins that typically need to be soluble in aqueous solvent to have an effect on animals such as humans. The invention also overcomes the uncertainties associated with delivery of agents to embryos through maternal feeding.

Embryos are permeabilized with a diluted EPS solution. EPS is a combination of a non-toxic cyclic terpene, for example, a plant derived cyclic terpene, such as limonene, particularly D-limonene, in combination with one or more non-toxic surfactants, preferably at least two surfactants, preferably two non-ionic surfactants. The surfactants can be present in various ratios relative to each other. Surfactants account for about 10%-30% of the volume of EPS, and the remaining volume is provided by the cyclic terpene. The EPS is a stable emulsion. After mixing the cyclic terpene with the surfactants, the mixture does not visibly separate preferably for at least about 24 hours.

In a preferred embodiment, the surfactants in the EPS are naturally derived surfactants, e.g., from plants such as coconut (e.g., coconut diethanolamine (DEA)). In an embodiment, composition of surfactant constitutes equal portions of DEA and ethoxylated alcohol together totaling 10% of the mixture. The DEA has a molecular weight of about 280-290 g/mol. In an embodiment, the DEA has a critical micelle concentration of about 1.48 g/L. The ethoxylated alcohol has a molecular weight of about 280-600 g/mol.

The EPS working solution is a dilute solution of EPS. Although the EPS stock solution is a stable emulsion, the dilute EPS working solution need not be a stable emulsion. In a preferred method, the agents to be tested for toxicity are soluble in aqueous media, preferably cell culture media in which the embryos are placed after permeablization. In some embodiments, the EPS working solution can include additional components to maintain the stability of the embryo after removal of the waxy layer. EPS can be diluted in a solution containing buffer and or salt to maintain the embryos at a physiological pH or salt concentration. EPS can be diluted in a solution including a non-active carrier protein, e.g., bovine serum albumin (BSA), ovalbumin (OVA). It is preferable that carrier proteins are larger than would be expected to pass through the embryo even after permeabilization. Similarly additional surfactants can be added, particularly high molecular weight surfactants to a working solution of EPS.

In the methods of the invention, treatment of embryos with a working solution of EPS occurs after the removal of the chorion layers. Although this step is routinely performed using a 50% bleach (3% sodium hypochlorite) solution, the EPS working solution treatment step can be carried out after any method used to remove the chorion layers.

The specific dilution of EPS to be used in any particular method is a matter of choice of one skilled in the art. For example, some strains of Drosophila may have less stable embryos than others after treatment with a working solution of EPS. Therefore, such embryos can be treated for less time, or with a more dilute solution of EPS. Conversely, as later developmental stages of embryos are harder to permeabilize, less dilute EPS and/or longer incubation times can be used.

Similarly, the amount of time that the embryos are contacted with the working solution of EPS can vary depending on the agents to be tested for toxicity or other properties. For example, embryos can be contacted with EPS for about 10 seconds to about 30 minutes, for about 30 seconds to about 20 minutes, for about one minute to about 10 minutes, for about one minute to about 5 minutes, for about 90 seconds to about 150 seconds. For example, a more concentrated working solution of EPS (e.g., 1:5 dilution) may require less time to permeabilize embryos to the desired extent than a more dilute solution (e.g., 1:50 dilution). Working embodiments are provided herein demonstrating times and concentrations required for the permeabilization of embryos to specific dyes. Experiments to test uptake of dyes or other detectable markers of known structure and size are routine. Method to vary time of exposure to working EPS solutions or various dilutions of working EPS solutions followed by observation to detect uptake of the detectable marker is well within the ability of one of skill in the art. Similarly, one of skill in the art can readily identify specific cyclic terpenes and surfactants that can be used in the methods of the invention. Optimization of concentrations of various components is within the ability of those of skill in the art.

It is understood that the methods for the invention can be scaled to accommodate the desired number of agents to be tested. For example, low-throughput methods can be performed using individual devices of the invention, manual pipettors, photomicroscopy, etc. Alternatively, high-throughput methods including the use of robotics and automated image collection and/or analysis is within the scope of the invention. These include adapting the procedure to a high throughput format, automating endpoint analyses such as image capture and analysis, and standardizing outcomes, determining sensitivity and validating the assay for regulatory applications. Instrumentation suitable for embryo sample handling in a 96-well, 384-well, or higher density plate format and image capture and analysis is commercially available (Union Biometrica COPAS SELECt™, Holliston Mass., The BD Pathway 855 Spinning Disk confocal system, BD Bioscience, San Jose, Calif.). Such modifications are within the ability of those of skill in the art.

### Embryo Incubation Device for Facilitation of Embryo Handling and Development.

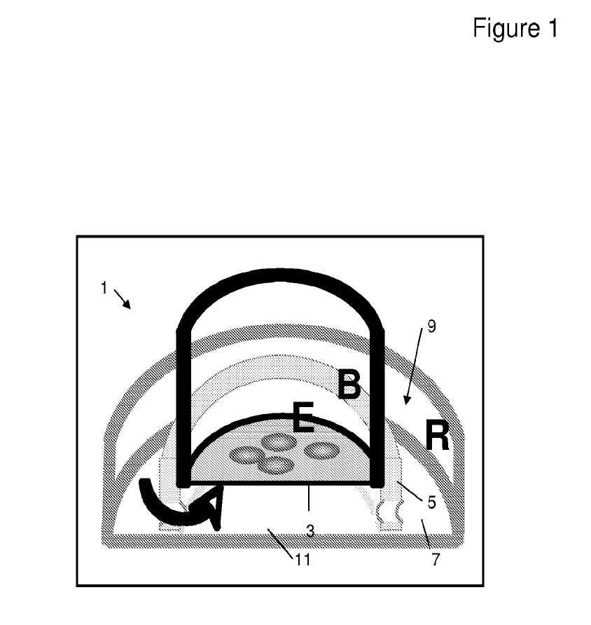

One of the first challenges of developing this method was to establish in vitro conditions for optimal embryo viability subsequent to treatments that affect the eggshell. We devised a chamber that was optimal for immersion of the embryo in surrounding buffer while maximizing oxygen delivery. We modified a conventional home-made “nitex basket” used for the 50% bleach treatment in the dechorionation step (FIG. 1). These baskets are typically fabricated from the screw cap of a 50 mL conical culture tube and the cut off top of the tube. The center portion of the cap is excised such that a patch of nitex screen can be screwed in place between the cap and the tube. When inverted a “basket” is formed that can effectively retain embryos when dipped in a reservoir or washed from above. We modified the basket by forming four notches in the rim of the cap such that when the basket is placed a reservoir solutes can flow and exchange freely across the nitex (see FIG. 1). We find that when placed in a reservoir of 6 mL of buffer in a 50 mm plastic dish the buffer is retained from entering the basket due to surface tension in the nitex pores, while the reservoir level on the outside of the basket raises above the level of the nitex. Embryos placed on the nitex in the basket are seen to become ⅔ immersed in the buffer due to capillary action. When basket and reservoir are capped with an inverted beaker reservoir evaporation is minimized and the embryo orientation in the fluid can be maintained for >48 hours. In our previous studies of methylmercury toxicity we have shown that drug delivery can be achieved by inclusion in the reservoir buffer. The notches in the rim also facilitate removal of air bubbles from the underside of the nitex while allowing solutes to diffuse freely within the bulk reservoir.

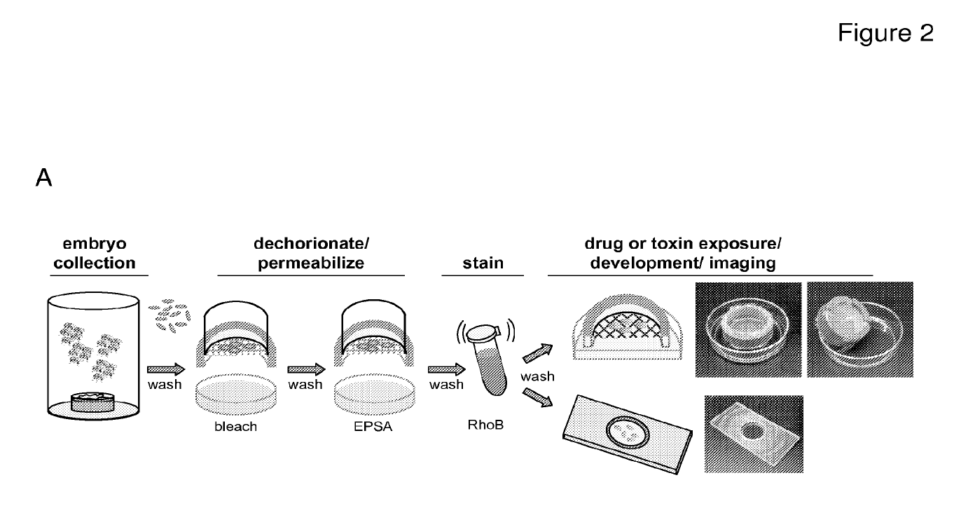

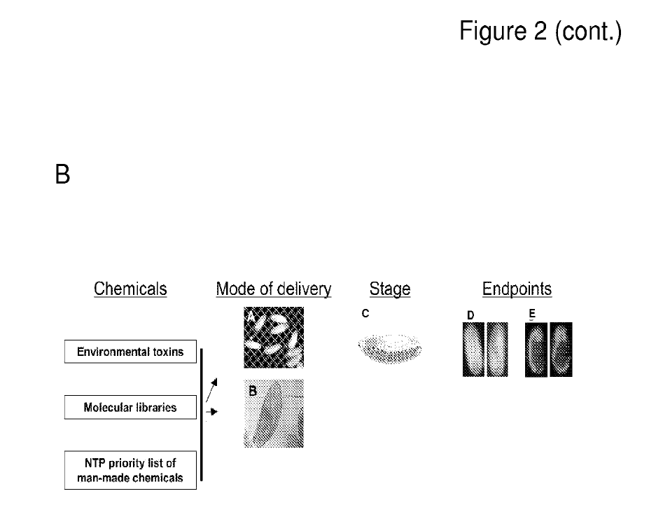

Alternatively, dechorionated and permeblized embryos can be arranged on slide chamber (see FIG. 2A). This slide allows for retention of embryos in a 0.3 mm layer of medium between a glass coverslip and a DO membrane (Fondriest Environmental, YSI #5793) that facilitates oxygen delivery. This set up is optimal for time lapse imaging of live embryos using a microscope with confocal capabilities.

FIG. 1 is a schematic of an embryo incubation device useful for practicing the methods of the invention, particularly for sequentially contacting the embryos with a series of solutions. However, the invention is not limited by the specific device used to perform the methods of the invention.

The device includes side walls 1, a mesh bottom 3, and a base support 5. The mesh is appropriately sized (˜60-120 μm grids) to retain the embryos, but also to allow fluid to pass through, or be wicked through the openings, as desired. In some embodiments, devices with different size mesh can be made, with larger openings for sodium hypochlorite treatment and washing, and smaller openings for treatment with permeabilization solution. In all cases, the mesh is sufficiently small that the embryos cannot readily pass through the mesh.

The base is configured so that the mesh bottom does not rest on the surface 7 on which the device is placed, and sufficiently weighted to keep the device upright when the solution is changed in the chamber 9 in which the device is sitting. Alternatively, the device can attach to the bottom surface of a chamber to keep it in place when the solution is changed in the chamber. The base includes at least two openings below the level of the mesh bottom, to let in fluid and to let out air, to allow liquid to be introduced into the space below the mesh bottom 11. Alternatively, the base can instead have at least three legs, which allow the device to stand stably on the bottom surface of a chamber, and the openings are the spaces between the legs. The surface of the chamber can also include appropriately shaped indentations or other forms to allow for the surface of the chamber to receive the legs of the base. Embryos 13 can be placed on top of the mesh surface. The device can further include a lid.

The devices are made of water-resistant material, such as plastic, and can be washable or disposable. The devices can further include a chamber into which solutions can be introduced. The chamber can be sized to hold one or more of the devices of the invention.

### Drosophila Embryo Structure and Methods of Agent Delivery

The invention includes methods for rendering the Drosophila embryos accessible to agents that would not typically pass through the waxy layer of the embryo for the purpose of characterizing the biological/toxicological activity of such compounds and their effects on embryonic development. The agents are preferably soluble in an aqueous buffer and/or culture medium suitable continued development of the permeabilized embryo. This invention makes possible a wide array of analytical test of toxicology and biological activity in the Drosophila embryo where this was not previously possible. The method disclosed herein overcomes a longstanding barrier to utilizing Drosophila embryos for such screening, namely, making the waxy layer of the embryo shell permeable to aqueous solutions while allowing the embryo to continue to develop. The compositions and methods provided herein open the door to using this important model for investigation of the vast number of man-made and naturally occurring compounds for which there is no biological or toxicological data.

Drosophila embryonic development has been documented with exceptional detail at the cellular and molecular-genetic levels. Furthermore, a majority of the fundamental cellular and molecular mechanism in embryonic organogenesis are conserved between Drosophila and mammals making fruit flies a highly relevant model for investigations of the potential impact of chemical compounds on human health and development.

The Drosophila embryo is enclosed in a shell made up of five layers: the innermost vitelline membrane, the waxy layer, the inner chorion, the endochorion and the outermost exochorion (FIG. 4A). The invention includes the use of a non-toxic, cyclic terpene, e.g., limonene, particularly D-limonene, in combination with at least one non-toxic surfactant, for removal of the waxy layer, the barrier that makes the embryo impermeable. D-limonene is a moncyclic monoterpene that is a major constituent of several citrus oils which can be isolated in bulk from orange peels. D-limonene is essentially non-toxic and is used widely as a fragrance and flavor additive in a number of foods, beverages and personal care products.

The methods of the invention include sequential steps for removal of the chorion and waxy layers, respectively. The embryo is then partially submerged in aqueous solution that permits access of small molecule solutes to the embryo by diffusion through the porous vitelline membrane. Oxygen delivery is maximized by exposure of the top surface of the embryo to air. Embryo development is monitored at the desired endpoint with the aid of a microscope or other imaging device. An example of a typical preparation is described below in the Examples.

At any point subsequent to agent exposure, the embryo can be processed for evaluation of the development of specific organ systems, e.g. nervous system, musculature, cardiac tissue, etc. These latter analyses can utilize a large array of techniques already in place for analyzing fly embryos, including immunohistochemical and fluorescent probe methods and confocal microscopy. Flies used can be any of the known fly strains including a vast array of strains that include mutations making the flies defective in one or more specific cell signaling pathways. A number of fly strains also exist in which a specific promoter is operably linked to a reporter gene facilitating the analysis of induction or inhibition of gene expression. The fly embryo has significant advantages over other model organisms such as C. elegans (roundworm) and zebrafish for the goals of molecular screening. While the fly embryo and C. elegans larvae are both readily adaptable to a 384 well format, fly embryos are not motile like the worm, which is advantageous for collecting static high-resolution fluorescence endpoint data. Although the zebrafish has the advantages of being a vertebrate and having organs and developmental processes more homologous to mice and man, maintenance of zebrafish carries considerably more expenses than either flies or worms. Furthermore, the shorter development time of the fly embryo (e.g. embryogenesis in 24 hours) enables a much higher throughput than with zebrafish. In essence, the Drosophila embryo consolidates many of the positive attributes of C. elegans and zebrafish for molecular screening in one organism.

This invention is different from existing methods of fly embryo permeabilization in that the permeabilization solution used is non-toxic and can be applied in an aqueous phase. Previous attempts to achieve permeability utilized organic solvents such as octane, which poses difficulties with embryo desiccation when going in and out of organic phase manipulations and introduces the inherent toxicity of the solvent.

Limonene forms an emulsion with surfactants and thus can be easily diluted in the aqueous phase (e.g., water, buffer) to make a working solution. An additional advantage of the methods provided herein is the ability to propagate embryo development subsequent to permeabilization in an aqueous medium thereby facilitating consistent dosing of the chemical of interest. Previous methods required immersion of the embryo in mineral or halocarbon oil after permeabilization to prevent desiccation and permit successful development, which also limited the agents that could be tested to those soluble in mineral oil to lipid soluble agents. The embryo incubation device, compositions, and methods disclosed herein permit optimal solute and air delivery for development. In addition the apparatus can easily be adapted to facilitate subsequent embryo manipulations, i.e. fixation protocols and microscopy imaging.

### Toxicology Screening Using Drosophila.

Although methods for toxicology screening are discussed herein, the EPS permeabilization solutions and methods can be used for any method that includes delivery of an agent to a Drosophila embryo in which the agent would not previously pass into the embryo sufficiently or consistently to provide the desired outcome.

Attention is drawn to three areas where development and application of this Drosophila model might benefit toxicology the most: 1) optimizing sensitive endpoints for pathway-specific screening, 2) accommodating high throughput demands for analysis of chemical toxicant libraries, 3) optimizing genetic and molecular protocols for more rapid identification toxicant-by-gene interactions. While there are shortcomings in the Drosophila model, which exclude it from effective toxicological testing it certain arenas, the level of conservation of fundamental cellular and developmental mechanisms between flies and man are extensive enough to warrant a central role of the Drosophila model in toxicological testing of today.

The methods of the invention are particularly useful for testing of the activity of a combination of agents. For example, it may be desirable to determine if one agent has a protective effect against the activity of another agent. The instant invention allows for predictable delivery of agents to embryonic cells, either sequentially or in combination with each other. As most agents that can be readily dissolved in the embryo maintenance buffer should be expected to pass through the embryo after permeabilization with about the same efficiency, data are not confounded by the variable uptake of agents.

### Drosophila Development

Flies are cheap and easy to maintain. They are propagated in small (30-50 mL) vials or bottles on a simple solid food medium of cornmeal, molasses, yeast and agar. Under standard culture conditions at 25° C. generation time is 12-14 days from egg to adult. The life cycle of the fly is comprised of four distinct stages: embryo, larva, pupa, and adult. Each of these stages present unique opportunities to assess susceptibility of the developing fly to agents potentially having biological activity. Normal development of essentially every organ system in the fly has been documented in exceptional detail.

The embryo develops in a period of 24 hours at 25° C. The development of the nervous system during this time has been studied extensively. During this time neurogenesis and differentiation within discrete regions of the ventral neurectoderm give rise to a fully functioning nervous system capable of executing the motor and sensory behaviors of the larvae including foraging, chemo- and phototaxis. In the ventral nerve cord the position and timing of birth of each of the 30 individual neuroblasts (NBs) within each hemisegment has been mapped in great detail (Bossing et al. 1996; Doe 1992; Schmid et al. 1999). In addition, the architecture of the neurons and glia that are propagated from each of these NB lineages has been resolved ((Schmid et al. 1999) and at www.neuro.uoregon.edu/doelab/lineages/overview/grasshopper-entres.html), presenting a exceptional template for evaluation of fundamental defects in neurogenesis and neuronal and glial morphology.

The larval stage progresses over four days and is characterized by a period of growth punctuated by two molts and resulting in a 10-fold increase in body size. While neural cell numbers in the CNS also increase 10-fold during these stages, due to a secondary wave of NB proliferation, neurons and glia remain relatively undifferentiated (Truman and Bate 1988). The following 5-6 days of pupal metamorphosis is a dramatic period of tissue reorganization, culminating in the fusing together of the presumptive adult structures from the precursor imaginal disc tissues. In the pupa larval-derived CNS and PNS neurons undergo dramatic pruning and re-growth while newly born adult neurons migrate to their final position and elaborate their processes to succinct targets (Truman et al. 1993; Williams and Truman 2004).

The newly hatched adult fly rapidly acquires characteristic behaviors of flight, chemo-, photo- and geotaxis, foraging and mating. These well-documented developmental and behavioral aspects of Drosophila make it an especially informative and adaptable model to investigate a wide variety of toxicological endpoints relevant to human biology and behavior.

### Toxicology Assays

FIGS. 2A and B illustrate modes in which Drosophila can be used in toxicological assays. FIG. 2A provides a schematic of a method of the invention for permeabilization and toxin exposure steps. FIG. 2B provides an alternate schematic of the invention showing 1) the variety of chemical toxicants to be investigated; 2) the mode of delivery to the organism; 3) the developmental stage of the fly; 4) the endpoints to be measured in determining biological/toxicological effects.

Compounds and mixtures for which toxicological information is needed are varied ranging from longstanding environmental contaminants to libraries of molecules derived from combinatorial chemistry from various industries including pharmaceuticals.

The mode of delivery is an important consideration for getting toxicant exposure to the cells or organ of interest in flies. Previously, exposure to embryonic tissues could be achieved through maternal feeding or through injection or in vitro incubation. Maternal feeding is effective for embryo exposures but requires determination of dosage empirically due to the unknown characteristics of metabolism and delivery. Embryo injection methods have been employed for decades for gene delivery and have recently been optimized for high throughput screening protocols suitable for molecular library screens (Zappe et al. 2006). Chemical exposure by direct incubation of fly embryos has proven effective in several cases, yet has been limited by the technical difficulty of permeablizing the waxy layer in the shell. In some cases small molecules such as methanol have been shown to make it into embryos with minimal manipulations giving distinct neural developmental outcomes (Mellerick and Liu 2004). Larvae and adult flies are readily exposed to chemicals through dosages in the food medium. Injection methods have also been employed for compound delivery in larvae and adults (Bijelic et al. 2005; Dimitrijevic et al. 2004). Adult flies are also easily exposed to vapors and aerosols in a controlled environment (Parr et al. 2001). Methods to enhance shell permeability provided herein open the door to a variety of applications for embryos in toxicology assays.

### Features of the Fly Model

Drosophila has been regarded primarily as a model for the study of genetics. Indeed, some of the first principles of genetics, such as the chromosome theory of heredity and the mutagenicity of X-rays were elaborated in the fly (summarized in (Rubin and Lewis 2000)). The large polytene chromosomes isolated from the salivary glands served as a template for physical mapping of genes and were revealed through banding patterns and eventually via hybridization methods. Mutagenesis screens, that exploited the easily interpretable endpoints of lethality, or disrupted embryo, wing or eye morphology, rapidly advanced the knowledge of gene function. As well, these mutants propagated the unique, sometimes humorous, nomenclature for their respective phenotypes, such as Hedgehog, Wingless, and Notch, which have subsequently become synonymous with the underlying signaling pathway. This detailed knowledge of genetic and signaling pathways makes Drosophila an ideal organism for toxicological assays as this knowledge can be applied to developmental and behavioral screens. The availability of many strains of flies with defined phenotypes corresponding to genotypes allow for the determination of susceptibility of flies with certain mutations to various agents. Selection of appropriate strains of flies for testing is well within the ability of those of skill in the art.

The relatively simple genetic make up of the fly consists of four chromosomes encoding approximately 13,600 genes, roughly half the number in humans (Adams et al. 2000). More than 95% of its genetic content is on the sex (first) chromosome and two autosomes (II and III), which has made conventional genetic analyses straightforward.

The tools of the Drosophila trade are as comprehensive for genomic, proteomic, bioinformatic and functional molecular studies as any model organism currently in use. Important online starting points are the Virtual Library: Drosophila (ceolas.org/fly/) and Flybase (flybase.org/). The Virtual Library: Drosophila contains a number of informative links to sites covering topics ranging from a basic introduction to Drosophila to specific neurobiological resources. Flybase (the database of Drosophila genes and genomes) contains annotations of all known genes including information on related mutant phenotypes, published and unpublished references and links to reagent sources.

### Examples of Measurable Endpoints in Drosophila Toxicology.

### Lethality:

Historically, Drosophila have been used in tests of genotoxicity. Lethality of adult flies subsequent to larval or adult chemical exposure has proven highly effective in analysis of heavy metals and, importantly, in screening for genetically encoded resistance traits (Christie et al. 1985; England et al. 1991; Magnusson and Ramel 1986). Over 20 years ago Magnusson and Ramos (Magnusson and Ramel 1986) utilized Drosophila in analyses of heavy metal toxicity, notably with inorganic mercury and methylmercury (MeHg). Importantly, their studies showed that tolerance to MeHg, as assayed by adult hatching after larval exposure, has a genetic component that displays a polygenic, yet dominant trait. Furthermore, this tolerance trait could be artificially selected for in the laboratory (Magnusson and Ramel 1986). While these studies were not continued to the level of isolating the genes involved, a similar experimental approach taken by the Jackobson lab found unique dominant traits in cadmium and strontium tolerance that resided on different chromosomes (Christie et al. 1985; England et al. 1991). Muniz Ortiz, et al. (Muniz Ortiz et al. 2008) have recently re-invoked this approach of using natural variation in tolerance and susceptibility to investigate mechanisms of arsenic toxicity. Calling upon contemporary methods developed since the Magnusson and Ramos studies, such as microsatelite recombination mapping, chromosomal deficiency lines and RNAi methodology, these investigators identified genes in the glutathione synthesis pathway that are functionally linked to arsenic tolerance (Muniz Ortiz et al. 2008).

### Inhibition of Larval to Adult Transition

The larval to adult transition in flies is complex and perhaps the least homologous developmental event with respect to mammals. As such, lethality assays of chemical exposure at this stage are not likely to return meaningful LD₅₀ values relative to mice or man for a given agent. Nonetheless, adult lethality remains a powerful screening tool for tolerance or susceptibility to a given chemical or genetic stress and thus has many applications to toxicological investigations.

The permeabilization methods of the invention allow for the more widespread use of flies for toxicology screening by providing a method for delivery of agents that would not normally be able to cross the waxy layer of the embryo without significant manipulation of the embryo (e.g., microinjection). The methods of the invention facilitate the use of high throughput screens which are particularly useful for screening for toxicity.

### Adult Morphology

Eye and wing phenotypes are a hallmark of Drosophila assays. For genotoxicity the somatic mutation and recombination test (SMART) continues to provide sensitive, rapid and inexpensive assays for mutagenic and recombinogenic properties of chemicals (Graf et al. 1984; Rizki et al. 2006). The SMART is based on the principle that the loss of heterozygosity through mutation or recombination of suitable recessive marker genes can lead to the formation of mutant clones of cells appearing as “spots” on the wings or eyes of the adult flies (Graf et al. 1984). The test requires treatment of larvae with the candidate compound and subsequently examining spot frequency in the adult tissue. The SMART test is useful in investigation of both mutagenic and anti-mutagenic properties of compounds, the latter being conducted in the combination with a proven mutagen and assaying for suppression of mutagenic activity (Abraham 1994; Rizki et al. 2001).

### Direct Observation

Direct assessment of teratogenic effects of chemicals in flies has been employed in a limited number of studies. The merit of this approach is the fact that a number of cell signaling mechanisms that direct development also act in a mechanistically conserved manner in morphogenesis of the wing and eye. Thus, an abnormal endpoint, such as wing notching, can indeed reveal an activity of the xenobiotic in question upon the Notch pathway. The approach of assessing teratology in the larval-adult transition could be enhanced by the rationale used in conventional enhancer/suppressor screen for genetic analyses. In these screens flies carrying mutations that partially debilitate function in a known signaling pathway become exceptionally susceptible to chromosomal and extra-chromosomal influences that alter signaling in that pathway. The potential for this approach in future assay development is discussed below.

### Behavioral Traits

Flies exhibit a wide array of behaviors relevant to understanding human response to environmental challenges. These behaviors include circadian rhythm, sleep patterns, courtship and mating, aggression, grooming and flight orientation. Many of these are under control of genetic and molecular mechanisms that were first resolved in Drosophila (Greenspan and Dierick 2004; Sokolowski 2001). Furthermore, at a physiological level the underlying neurotransmitter systems in the fly are conserved including serotonin, dopamine, GABA, glutamate and acetylcholine (reviewed in (Nichols 2006)).

To date, behavioral endpoints in Drosophila have been used primarily to isolate genes that specifically support a given trait versus a tool for screening vast numbers of chemicals. This is exemplified by the initiative to find genes that regulate ethanol (EtOH) tolerance which lead to identification of the “cheapdate” mutant (Moore et al. 1998). The cheapdate mutant, which is a defective allele of the fly pituitary adenylate cyclase activating peptide (PACAP) homolog known as amnesiac, was one of 12 mutants identified from screening ˜5000 mutant alleles. Further genetic and molecular analysis has resolved that activation of the cAMP signaling pathway is central to the behavioral response to EtOH intoxication, not only in flies but in mice (Maas et al. 2005). Thus, in this example, a defined behavioral trait in flies has fostered dissection of a conserved toxic mechanism for ethanol. Courtship, fecundity and locomotion are reliable behavioral outputs that respond to toxic substances, such as lead in larvae (Hirsch et al. 2003).

### Embryonic Development

Fly embryos are highly effective for in vitro analysis of developmental toxicity. Capitalizing on the developmental potential of early gastrulating embryos the Bournias-Vardiabasis lab has devised an in vitro cell culture assay to evaluate teratogenicity (Bournias-Vardiabasis and Teplitz 1982). Cells from homogenized 3.5 hour old embryos subsequently cultured for 24 hours display a quantifiable degree of differentiation revealed by the formation of two structures: neuronal ganglia and myotubes. These structures, once highlighted by fixation and staining, are quantified with an automated image capture and analysis system. Assay of potential teratogenicity is revealed by a decrease in the number of one or both of these differentiated cell structures. The instant method allows for the observation of development of all structures in an intact embryo.

### Reporter Gene Assays

An example of the utility of transgenic reporters in fly toxicology comes from the Chowdhuri group, who have made extensive use of a transgenic fly carrying a lacZ reporter gene under the control of the heat shock protein 70 promoter (hsp70-lacZ) (Mukhopadhyay et al. 2003). Hsp70 is a highly responsive stress gene with fairly low specificity with respect to environmental stressor. Nonetheless, this reporter has served as a rapid readout of toxicity in larval and adult feeding assays of specific organophosphates as well as complex mixtures of industrial leachates (Gupta et al. 2007; Siddique et al. 2008). In addition, immunohistochemical analysis of the reporter reveals tissue specific patterns of toxic insult informative for sex-linked differences in response (Gupta et al. 2007). As with the embryonic cell assays mentioned above, application of more specific promoter-based reporter genes in transgenic flies to toxicological screens could prove highly effective and efficient for identifying chemical-pathway interactions in vivo. Methods for generating flies with transcriptional regulatory regions operably linked to reporter genes is within the ability of those of skill in the art.

### Targeted Mechanistic Studies

The fly model has also been implemented in efforts toward managing toxicity in drug administration. Methotrexate (MTX) is an anti-folate drug commonly used in chemotherapy due to its inhibitory action on dihydrofolate reductase (DHFR) and the subsequent lethality imposed on rapidly dividing cells. Its use has expanded to treatment of rheumatoid arthritis, asthma, Crohn's disease and lupus as well as in termination of early pregnancies (Domenech et al. 2008; Hedman et al. 1996; Ostensen et al. 2000; Wong and Esdaile 2005). MTX elicits skeletal defects and developmental abnormalities in the fetus, which conflict with its use in chemotherapy.

The Walker lab has exploited the high degree of conservation of DHFR and folate metabolism in the fly to screen for MTX-resistant variants of DHFR in a laboratory selection paradigm (Affleck and Walker 2008). Interestingly, MTX elicits analogous defects on gestation and embryogenesis in the fly, causing reduction of egg production in females with corresponding deformities in the ovaries and a variety of embryonic defects in the progeny (Affleck et al. 2006). These investigations have lead to identification of a L30Q mutant that confers high resistance to MTX in vivo in flies (Affleck and Walker 2007). Mutation of the homologous amino acid in mammalian DHFR shows MTX resistance as well (Dicker et al. 1989; Meisel et al. 2003), thus establishing a complete model of functional homology. Future efforts will exploit the fly system to screen additional MTX-resistant variants and dissect the mechanism of DHFR-independent MTX toxicity, all of which advance novel strategies for avoiding unwanted MTX toxicity in a clinical setting (Wolfgang 2007).

### Kits

The invention includes kits containing one or more devices or reagents to practice the method of the invention with appropriate packaging and/or instructions for use. For example, a kit can include any combination of an embryo incubation device, either a single device or a device with multiple wells for treating multiple wells of embryos simultaneously, a container of EPS, a container of bleach (either as a concentrated or working solution), a buffer for incubation of the embryos after EPS treatment, one or more dyes of different sizes and/or structures to confirm the efficacy of EPS treatment, control agents, and instructions for use.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991); Antibody Phage Display, (O'Brien and Aitken, 2002); Fly Pushing (Greenspan 2004), Drosophila Protocols (Sullivan et al., 2000). These techniques are applicable to the production of the polynucleotides and polypeptides of the invention, manipulations of Drosophila, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

This invention is further illustrated by the following examples, which should not be construed as limiting.

### EXAMPLES

### Example 1

### Permeabilization of Embryos with EPS

A healthy stock of flies containing 300 or more mated female flies were maintained in population cage. The cage was outfitted with an embryo collection plate consisting of a solid grape juice/agar mixture in a Petri dish seeded with yeast paste to prompt laying. Embryos were collected within a short time frame (e.g. 1-4 hours) to obtain embryos of similar developmental stage. The embryos were harvested from the plates by washing them into an embryo incubation device (FIG. 1) fabricated from the inverted top and cap of a 50 mL polypropylene disposable test tube. The bottom of the basket was made of 120 μM Nitex® or other similar mesh that retains the embryos, but allows flow-through of water or solvent when immersed at sufficient depth in the reservoir (see FIG. 1).

Embryos were washed extensively by dipping the basket in a reservoir of tap water with several subsequent changes of the water. The chorion layers were then removed by a standard protocol of immersing the basket of embryos in 3% sodium hypochlorite (50% bleach) for 2 minutes with gentle agitation. The embryos were then washed by repeated dipping of the basket in water as described above. This treatment was effective in removing 100% of the chorion and exposing the waxy layer.

As a first approach to remove the waxy layer, we evaluated the ability of the common household product Citrasolv® (Citra-Solv, Danbury Conn.) to render dechorinated embryos permeable. Citrasolv® consists of limonene, essential oils and biodegradable cleaning agents derived from coconut. To assess permeability we incubated embryos treated with Citrasolv® in histological dyes of various molecular weights. In initial experiments dechorionated embryos were immersed in a 1:10 dilution of Citrasolv® and water for five minutes, rinsed in PBS and immersed in 0.1% solution of Dye for 30 minutes to assess permeability.

### Example 2

### Preparation of a Defined Embryo Permeabilization Solvent (EPS) Formula

Citrasolv® is composed of predominantly limonene and a proprietary (i.e., undisclosed) mixture of surfactants and oils. We therefore formulated a limonene/surfactant mixture of known composition. We surveyed the ability of several surfactants to render limonene miscible with water, as well as the ability of the diluted mixture to permeablize dechorinated embryos. An optimal mixture consisted of 90% D-limonene, 5% nonionic ethoxylated alcohol and 5% coconut diethanolamide. We termed this defined composition EPS (embryo permeablization solvent). EPS proved to be equally effective as Citrasolv® in rendering embryos permeable to dyes.

Limonene alone is not miscible with water. The presence of at least one surfactant is required to allow for the formation of a stable emulsion for sufficient time to allow for permeabilization of the embryos (i.e., at least about 10 minutes). Dechorionated embryos exposed to a mixture of 9% limonene with water (without surfactant) for 2 minutes showed variable penetration of Syto11®. The pattern showed spotted regions of penetration in to embryonic tissue suggesting a non-uniform action of the limonene at sites on the waxy layer. Similar results were obtained with 1:10 dilutions of 90% limonene/10% DEA as well as 90% limonene/10% ethoxylated alcohol. A uniform penetration of Syto11® was seen with compositions containing two surfactants (e.g. 90% limonene/5% DEA/5% ethoxylated alcohol). As well, working solutions omitting the limonene and containing the surfactants alone at the relevant concentration yielded no penetration of Syto11®.

Embryo permeabilization solvent (EPS) used in the experiments described herein consisted of 90% D-limonene (Technical Grade D-limonene the Florida Chemical Co. or high purity orange terpene D-limonene from Greene Terpene, www.greenterpine.com, eoilco, South Miami, Fla.), 5% cocamide DEA (Ninol® 11CM, Stepan Chemical) and 5% ethoxylated alcohol (Bio-Soft® 1-7, Stepan Chemical). This composition was stable at room temperature for at least a month. Additional formulations examined include substitution of limonene with terpenes from grapefruit, eucalyptus and peppermint (from Green Terpene). As well, the surfactant Biosoft® N91-8 was substituted for Biosoft® N1-7, and the DEA substitute Masamide R-4 (Mason Chemical Co. Arlington Heights Ill.) was examined. In addition, the limonene concentraion was reduced by substitution with mineral oil up to 45%. Dechorionated embryos were immersed in various dilutions of EPS in Milli-Q® water. A typical treatment utilized a 1:10 dilution of EPS in water with a one-minute exposure of 3-hour old embryos. Variations of treatments ranged from 1:1 to 1:20 dilutions of EPS in water and 15-second to 5-minute immersions. EPS treatment was followed by six successive washes in 5 mL of either Phosphate Buffered Saline (PBS, 139 mM NaCl, 2.7 mM KCl, 8.1 mM Na₂HPO₄, 1.5 mM KH₂PO₄, pH 7.0) or modified D22 medium at 0.8× concentration (0.8D22, 4.6 mM potassium glutamate monohydrate, 42.8 mM sodium glutamate monohydrate, 66.7 mM glycine, 4.4 mM MgCl₂, 13.7 mM MgSO₄, 2.75 mM NaH₂PO₄, 7.2 mM CaCl₂, 0.17 mM Na-acetate trihydrate, 0.47 mM succinic acid, 4.5 mM malic acid, 10 mM glucose (no lactalbumin or yeastolate), pH6.7 [11].

### Example 3

### Culturing of Permeabilized Embryos and Agent Delivery

After dechorination and permeabilization, the embryos were cultured by immersion of the basket in a reservoir containing a buffered solution that is isotonic with the embryo or in a slide device shown in FIG. 2A. A useful buffer formulation is D22 medium (commercially available from a number of sources, e.g., US Biological, described by Eschalier (Eschalier, 1997)) and is used at 4:1 dilution with water to match isotonicity of the embryo. The reservoir level was adjusted to make the buffer adhere to, but not penetrate, the underside of the mesh. Channels in the underside rim of the basket allow for free exchange of solutes from the reservoir to the undersurface of the mesh (see arrow, FIG. 1). The capillary action of the Nitex® mesh and surface tension of the buffer facilitate wicking of the solution around each embryo to a stable point at which approximately two-thirds of the embryo was immersed in the solution and one-third was exposed to the air (FIG. 1).

### Example 4

### Delivery of Agents to Permeabilized Embryos

In the experiments described herein, desired chemical compounds were added to the reservoir at various concentrations to effectively introduce a defined dose of the chemical of interest to the embryo.

Incubations of permeabilized embryos were carried out in two types of chambers. Longer-term incubations (6-48 hours) were done in a modified nitex basket (see FIG. 1). The basket-reservoir apparatus was covered with a tight fitting cap to limit evaporation and was incubated at 18° C. for continued development of the embryo. Short term incubations <3 hours were done in a slide chamber (see FIG. 2A). Incubations were done in either 0.8D22 buffer or PBS with the same effectiveness of developmental outcomes. Incubations for early embryonic endpoints were done at room temperature and for later developmental endpoint (e.g. gut development and engrailed patterning) at 18° C. Development to the larval stage is complete in 36-48 hours depending on the age of the embryo at initiation of treatment.

Drug treatments were done by additions to the PBS solution reservoir during incubation in either the slide or basket chambers. Drugs used were: cycloheximide (Fluka 01810, MW=281.35, 50 mM stock in H₂O), colchicine (Biomol Labs T118, MW=399.0, mM stock in H₂O), methylmercury chloride (Aldrich 442534, MW=251, 50 mM stock in DMSO), cytochalasin D (Sigma C8273, MW=507.6, 5 mM stock in DMSO) and cyclophosphamide (LKT C9609, MW=279.1, 100 mM stock in H₂O).

### Example 5

### Microscopy of Permeabilized Embryos

### Light and Fluorescence Microscopy

Imaging of embryos was done with three microscope setups: a Nikon® SMZ1500 stereo dissecting microscope equipped with a xenon light source for fluorescence imaging in the blue, green and red channels; a Leitz® Orthoplan 2 upright microscope equipped with Nomarski (DIC) optics and fluorescence capabilities; a Nikon® C1 scanning laser system on a Nikon® Eclipse E800 upright microscope using 10× and 20× objectives. Images were captured with a Spot One digital camera and associated PC Spot One software (MVI Avon Mass.). Confocal images were collected using the associated Nikon® C1 software package. Image processing was done using Adobe® Photoshop for static images and Image J software for time-lapse series.

### Electron Microscopy

Specimen preparation and scanning electron microscopy was performed with the help of the University of Vermont College of Medicine Microscopy Imaging Facility. Dechorinated Canton S embryos of mixed age (an overnight collection) were untreated or treated with EPS (no dilution 30 seconds) prior to fixation. A modification of Karnovsky's fixative was used (2.5% glutaraldehyde, 0.1% paraformaldehyde in 0.1M Phosphate Buffer (PB) pH7.2). Fixation was done for one hour followed by three rinses in PB. Post-fix was done with 1% osmium tetroxide in PB for one hour at 4° C., followed by three repeated washes. Dehydration was done in sequential 10-minute immersions in ethanol (35% up to 100% in six steps). Sample was then critical point dried in liquid CO₂. Samples mounted on an aluminum holder were sputter coated with gold/palladium for 2-4 minutes. Imaging was done on a JSM® 6060 scanning electron microscope (JOEL, Peabody, Mass.).

### Example 6

### Scoring of Embryo Development

Effects of EPS and cycloheximide or other agents on embryo development were scored using contemporaneous DIC optics and fluorescent visualization. Canton S embryos were collected from a 3 hr laying and aged one additional hour at 25° C. Embryos were treated with 1:10 EPS in H₂O for 30-60 seconds followed by washing and immersion in 1 mM rhodamine B dye in PBS for 5 minutes with rocking. Embryos were washed four times in PBS and immersed in PBS containing 10 μM cycloheximide on a Kiehart slide. Embryos were allowed to develop an additional 1.5 hours at ambient temperature (˜23° C.). Abnormal development was scored in embryos at Stage 7-9 that displayed absence or aberrant cephalic furrow formation and/or disrupted germband elongation. Variable treatments included omission of EPS exposure or omission of cycloheximide or other toxic agent. Each condition was assayed in three independent preparations of embryos with >180 embryos scored for each treatment condition.

### Example 7

### Dye Uptake in Embryos after Permeabilization with EPS

The ability of a dilute solution of EPS to permeabilize the waxy vitelline layer was tested. Freshly laid embryos were collected and washed with water and the chorion layers were removed by brief immersion in sodium hypochlorite as above, followed by thorough washing with water. In preliminary studies, embryos were then treated by immersion in a dilute solution of EPS (D-limonene, cocoanut DEA, Alcohol ethoxylate, typically 1:10-1:50 dilution in water for 2 minutes) and washed extensively with water. Permeability was assessed by immersion of embryos in various dye solutions in a standard phosphate buffer for a period of 30 minutes to 1 hour.

In later studies, dye uptake was assessed by immersing embryos in one milliliter of PBS or 0.8D22 buffer containing dye (at 0.1% or 1 mM concentration) for either five minutes in a capped microfuge tube rocking on a nutator or by immersion for 30 minutes in a Nitex® basket in a reservoir of dye. Dyes used were cresyl violet (0.1%, Sigma C5042, MW=321.3), Rhodamine B (1 mM final in H₂O, Sigma #R6626, MW=479.0), fast green (0.1% final, Fisher F99, MW=808.9), Syto11® (5 mM stock in DMSO, used at 5 μM final, Invitrogen S7573), Calcein® AM (1 mg/mL stock in DMSO, used at 5 μM final, Invitrogen C3099, MW=994.9). Embryos were then washed four times with PBS or 0.8D22 prior to visualization by brightfield and fluorescence microscopy. No significant differences were observed in the results from the two methods.

As seen in FIG. 4, three common dyes, (A, B) cresyl violet (mw=321), (C) fast green (mw=809), and (D) rhodamine B penetrated EPS-treated embryos to stain embryonic tissue (B-D). Note that the differences in (E) structure, net charge, and size of these dyes suggests a variety of compounds can gain access to the embryo. In the absence of EPS treatment there was no evidence of dye penetration, even with the lower molecular weight cresyl violet (FIG. 4A and data not shown).

### Example 8

### Electron Microscopy Demonstrates Removal of Waxy Layer

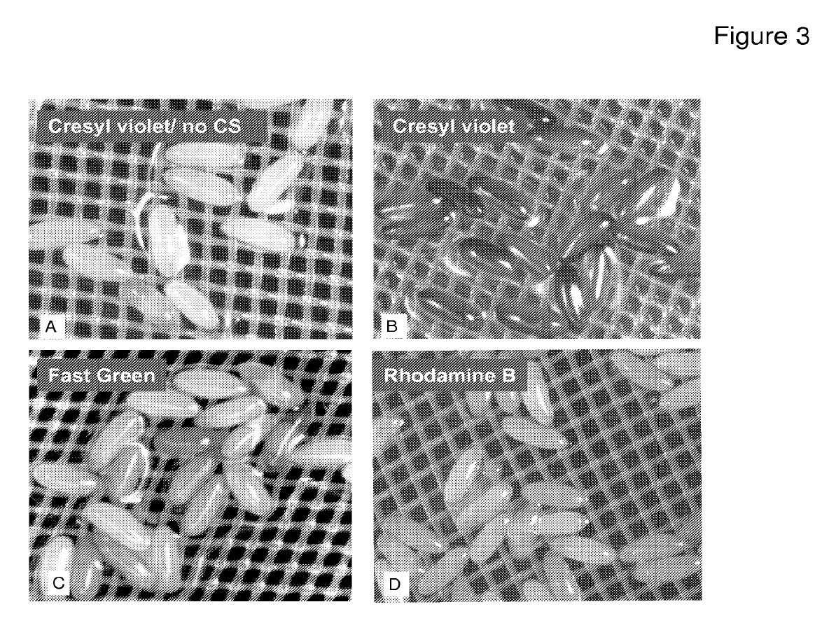

Having demonstrated uptake of dyes dependent upon treatment with EPS, we wanted to confirm that the treatment was removing the waxy layer as predicted. We assessed the ability of EPS to remove the waxy layer of dechorinated embryos directly using scanning electron microscopy (EM). The waxy layer is a very thin (5 μm) coating over the vitelline membrane. The waxy layer is formed by secretions from the follicle cells in the ovary and results in interleaved layers of flattened plaques of wax (FIG. 4A). This layer is removed through conventional fixation protocols for EM preparations. We therefore made preparations of dechorinated embryos, untreated and treated with EPS, omitting exposure to organic solvents and directly fixing in glutaraldehyde/paraformaldehyde and post fixing in osmium tetroxide. Scanning EM images of the control treated embryos revealed a roughened plaque-like coating of the surface consistent with the waxy plaques previously described (FIG. 3B, [18]). In contrast, EPS treated embryos showed a remarkably smooth and homogeneous surface, indicating removal of the waxy plaque layer and exposure of the vitelline membrane proper (FIG. 4B).

### Example 9

### Further Analysis of Dye Uptake in Embryos after Permeabilization with EPS

### Syto11® Green

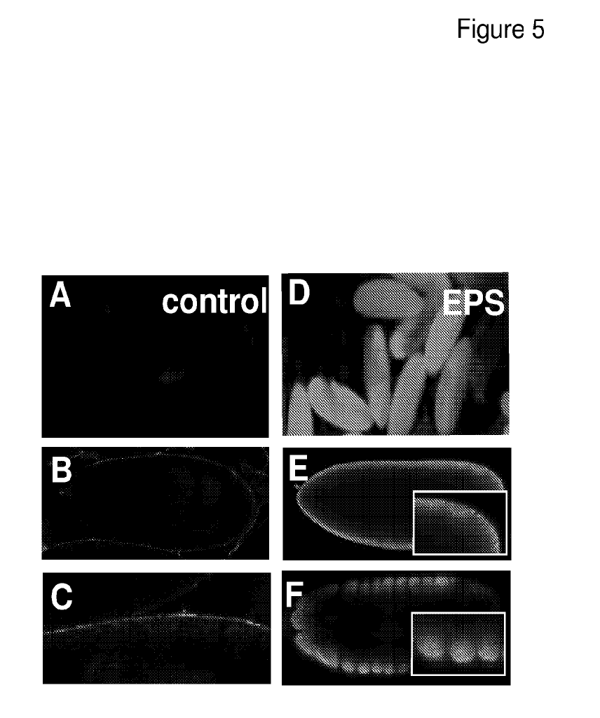

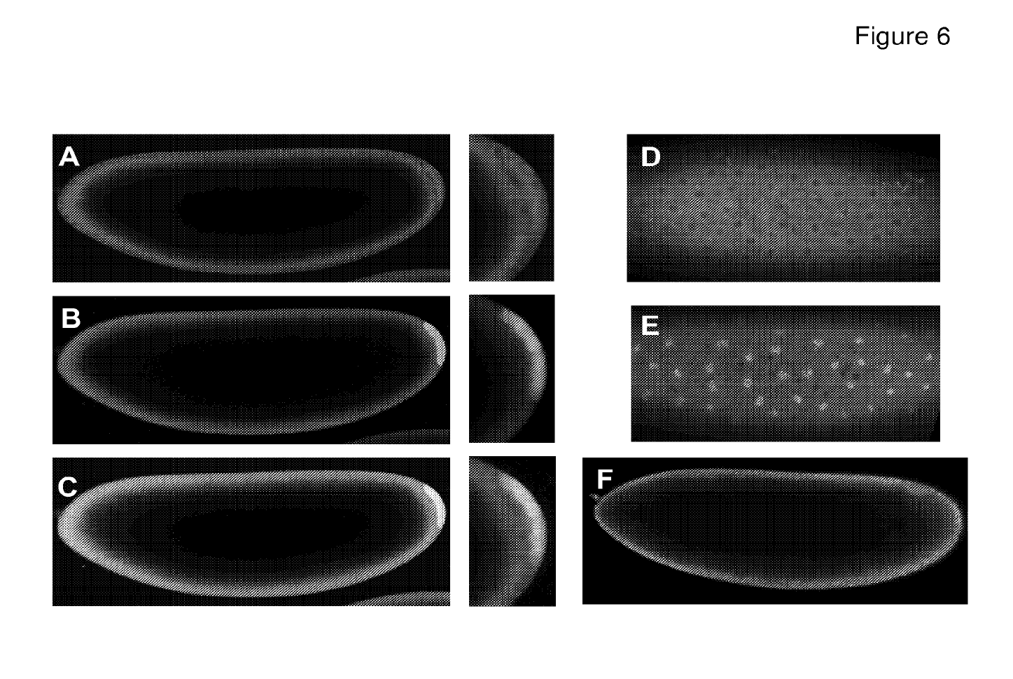

We examined the ability for compounds to penetrate the embryonic tissues using the Syto11® green fluorescent nucleic acid stain (Invitrogen, catalog #S7573). Syto11® is cell-permeant and displays a large fluorescent enhancement upon binding nucleic acids. Syto11® binds both DNA and RNA and exhibits staining of both cytoplasm and nucleus, however accentuates condensed chromosome structures during mitotic events. Staining of EPS treated embryos with Syto11® showed penetration of the dye, which is seen to light up cells and condensed DNA in the periphery of a Stage 6 embryo (FIG. 5E). Syto11® can penetrate several cell layers deep into embryonic tissues of a stage 11 embryo (FIG. 6F). No dye penetration was observed in cells not treated with EPS (FIG. 5A-C). Altogether, these data demonstrate that EPS treatment renders the embryo permeable and accessible to small molecules of molecular weight up to ˜1,000 Dalton. In addition, where such compounds are membrane permeable they are able to distribute to significant depths of the embryonic tissue in a relatively short time frame.

### Rhodamine B, Calcein AM, and Syto®11

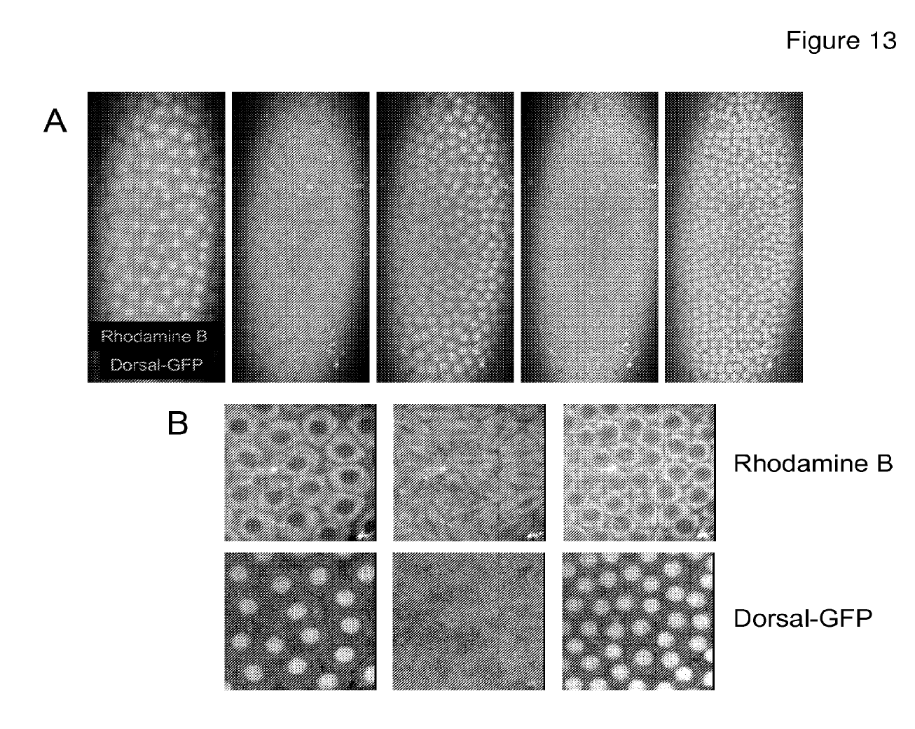

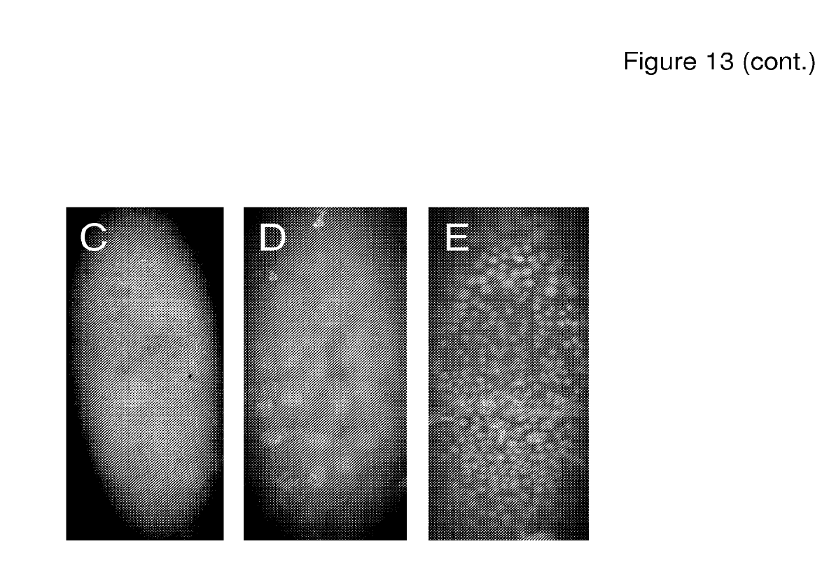

Rhodamine B is a brilliant red fluorescent dye that showed to accumulate in the cytosol of pre-blastoderm embryos as well as in the first cells formed at the blastoderm stage (FIG. 6A, C, D). This distribution is consistent with the reported property of endosomal uptake of rhodamine B. Remarkably we found that rhodamine B was excluded from the nucleus, and could be used to monitor mitotic stage of the cell through time-lapse confocal microscopy (See FIG. 6D and FIG. 13). Calcein® AM is a cell permeable dye that becomes fluorescent once cleaved by esterases in the cytosol of viable cells. Calcein is seen to label the cells in the periphery of a blastoderm embryo (FIG. 6B, C). Notably, pole cells in the posterior of the embryo are strongly labeled (FIG. 6B, C) presumably due to the more proliferative nature of these cells at this stage. Syto 11 is a cell permeable nucleic acid binding dye that is rendered fluorescent upon binding nucleic acids, notably chromosomal DNA. Remarkably, Syto® 11 is seen to strongly label the condensed chromosomes of pre-mitotic figures of an early embryo (undergoing nuclear division 13) (FIG. 4E). In addition, Syto® 11 uptake can be seen in the cells at the periphery of an embryo undergoing gastrulation (FIG. 6F). Together, these data demonstrate the efficacy of EPS to render the embryo shell permeable to a variety of dyes of various molecular weights and subcellular distribution properties.

### Example 10

### Analysis of Viability after Permeabilization

Previous attempts to render embryos permeable demonstrated an inverse relationship of permeability with viability. Nonetheless, careful manipulations using heptane demonstrated a narrow window of exposure to solvent that resulted in a significant number of development-competent embryos. These earlier studies employed rhodamine B as an indicator of permeability, however, dye uptake was only assessed at the light microscopy level. With the objective of establishing optimal conditions of permeabilization and embryo viability we investigated the effects of various conditions of EPS treatment on permeability and viability. We first observed that staining EPS treated embryos gave a highly heterogeneous uptake of the dye as seen by light microscopy with a standard dissecting microscope (FIG. 3), presumably reflecting a high degree of variability in permeabilization.

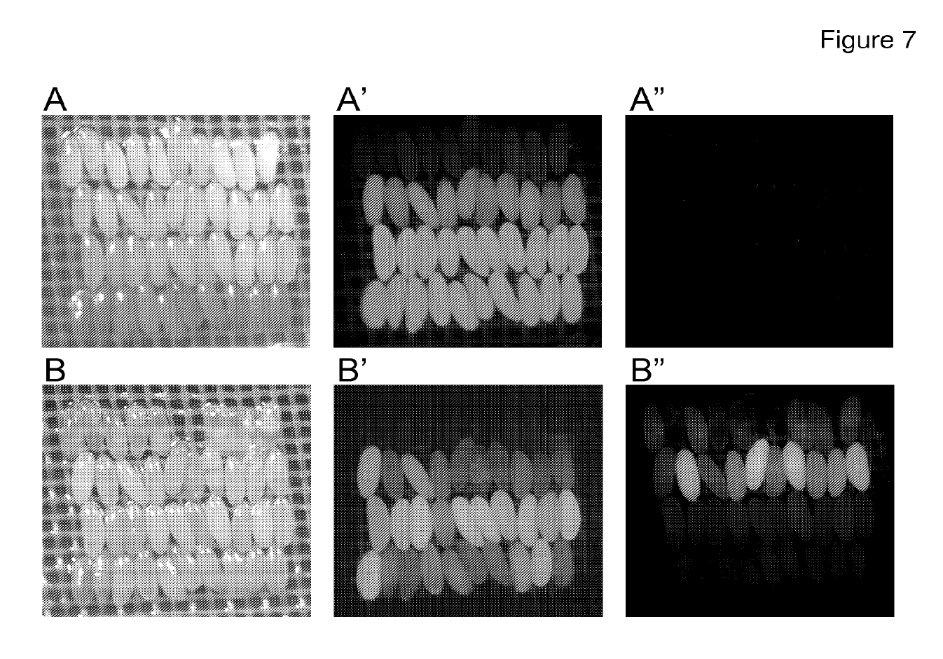

To assess developmental outcomes of these embryos we could effectively sort the embryos based on their degree of rhodamine B uptake. We then assessed the rhodamine B levels in these embryos by fluorescence. This demonstrated a much higher degree of sensitivity to detect the dye, showing that nearly white embryos under light microscopy display brilliant red fluorescence (FIG. 7A, A′). As well, embryos displaying essentially no red fluorescence proved to indicate impermeability. Embryos exhibiting a discernable pink/red color by light microscopy showed essentially saturating levels of bright red fluorescence (FIG. 7A, A′). Thus, assessing rhodamine B uptake by fluorescence proved to detect a much lower threshold of permeability as compared to rhodamine B visualization by light microscopy.

Allowing EPS treated and rhodamine B stained embryos to develop for a period of 18 hours at 18° C. demonstrated an overall inverse relationship of permeability and viability. Embryos with the lowest dye incorporation developed to late stages as seen by formation of internal organs under light microscopy (top row, FIG. 7A, B). Embryos with the highest dye incorporation failed to develop at all (Bottom two rows, FIG. 7A, B). As well, dye was seen to leach out of the strongest stained embryos over the 18 hr period (Bottom row, FIG. 7B). Notably, embryos with uptake of rhodamine B at an intermediate intensity showed development to late stages (Second row, FIG. 7A, B). An unexpected finding was that these embryos exhibited brilliant flourescence in the green wavelength after the incubation period, which corresponded with a reduction of corresponding red fluorescence (note second row in FIG. 7A′,A″, B′, B″). We predict that this results from metabolism of the rhodamine B dye causing a spectral shift in its flourescence. Rhodamine B (N,N,N′,N′-tetraethylrhodamine) is known to undergo de-ethylation which causes a shift in its excitation/emission properties (Qu, 1998). A lack of dye flourescence conversion was seen in the highly red-stained embryos, which is consistent with the embryo being overtaken by cell death due to excessive permeabilization.

### Example 11

### Dynamics of Rhodamine B Dye Uptake and Conversion in Permeabilized Embryos

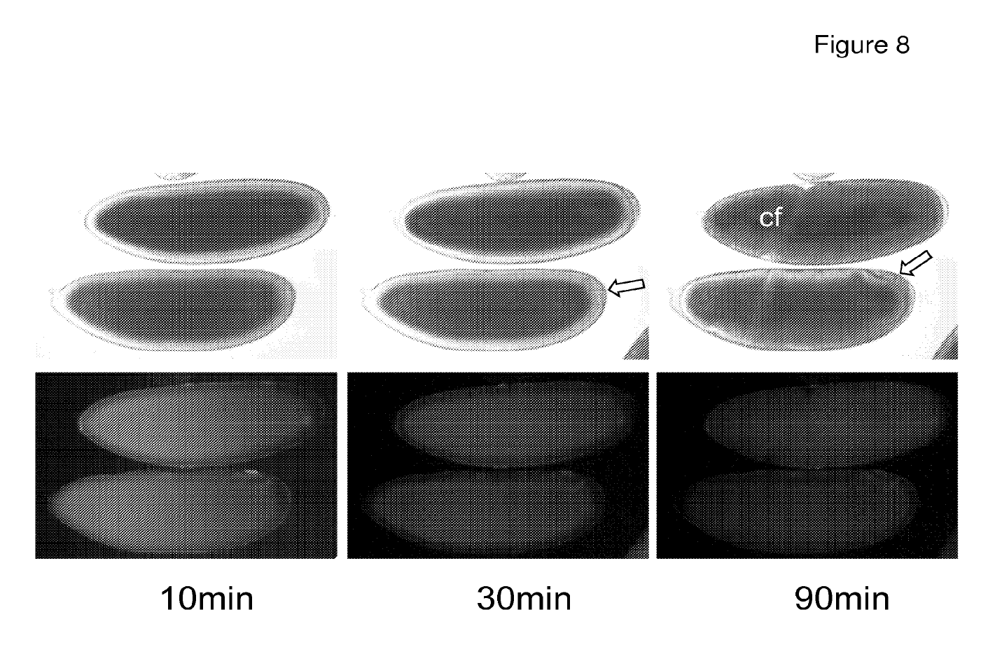

To investigate the response of rhodamine B dye in viable embryos we investigated this effect further by time-lapse imaging. First, we observed an anterior to posterior gradient of Rhodamine B distribution in embryos in the 10-20 minutes immediately following dye exposure. As seen in FIG. 8, at 10 minutes after dye exposure rhodamine B is most concentrated near the anterior and extends to the middle of the anterior-posterior axis. Rhodamine B becomes uniformly dispersed over the next 20 minutes and remains evenly distributed out at 90 minutes during which time the cephalic furrow has formed and pole cell migration has progressed (FIG. 8). A less intense overall red fluorescence is seen at 90 minutes presumably due to the conversion of rhodmine B to the green wavelength as eluded to above. These data suggest the anterior portion of the embryo is more susceptible permeabilization with EPS. In addition, an initial gradient of concentration can be expected over the course of administering drugs to the embryo at early time points and should be considered with respect to the timing of early developmental events.

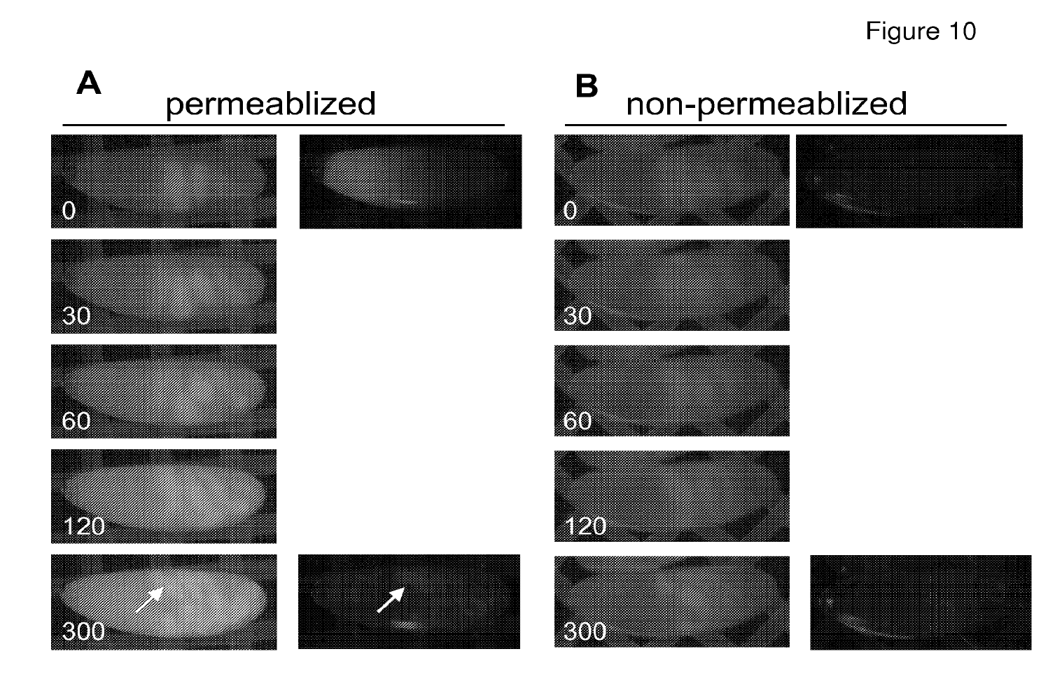

A closer examination of the differential uptake of rhodamine B among EPS treated embryos revealed that early stage embryos incorporated the dye more readily than older embryos. The developmental stages of the Drosophila embryo have been extensively documented (Hartenstein, 1993, incorporated herein by reference) and a condensed timetable is presented in FIG. 10A. Embryos harvested and treated in the first three hours of development (up to stage 5, preblastoderm stage) were efficiently permeabilized with a standard treatment of 1:10 diluted EPSA for 30-60 seconds (FIG. 6B). Dye uptake is much reduced and often undetectable in embryos older than Stage 11 undergoing the same EPS treatment (FIG. 6B). However, longer treatments and/or more concentrated EPS solutions can achieve permeability of later stage embryos (e.g. Stage 16 embryo in FIG. 10).

Embryos with an initial strong accumulation of rhodamine B dye demonstrated a steady increase in green fluorescence over a five hour incubation period with the majority of green fluorescence signal achieved in ˜2 hrs (FIG. 10). A corresponding loss of red fluorescence is seen over this time period (FIG. 10). As a control, an embryo of the same stage from the same preparation that was not permeabilized as determined by the absence of red fluorescence shows no evolution of green fluorescence over the same time period (FIG. 8B). Thus, we interpret this fluorescence conversion to reflect metabolic activity of viable cells in these embryos. It is also of note that a green auto-fluorescence is seen to concentrate in the gut in non-permeabilized embryos. This fluorescence derives from the residual yolk proteins that accumulate in the gut at late stages. It is of note that an elevated red fluorescence is observed in the developed gut of permeabilized embryos. This result is consistent with the notion that rhodamine B can accumulate in the gut where it is prevented from converting to the green-emitting fluorophore. Together, these data demonstrate that a degree of permeability can be achieved that is compatible with embryo viability. These data also indicate that the level of permeability can be effectively gauged initial “gradient” of rhodamine B uptake and subsequently by the elaboration of green fluorescence due to spectral conversion of rhodamine B.

### Example 12

### Developmental Endpoints for Scoring Drug Effects in Permeabilized Embryos

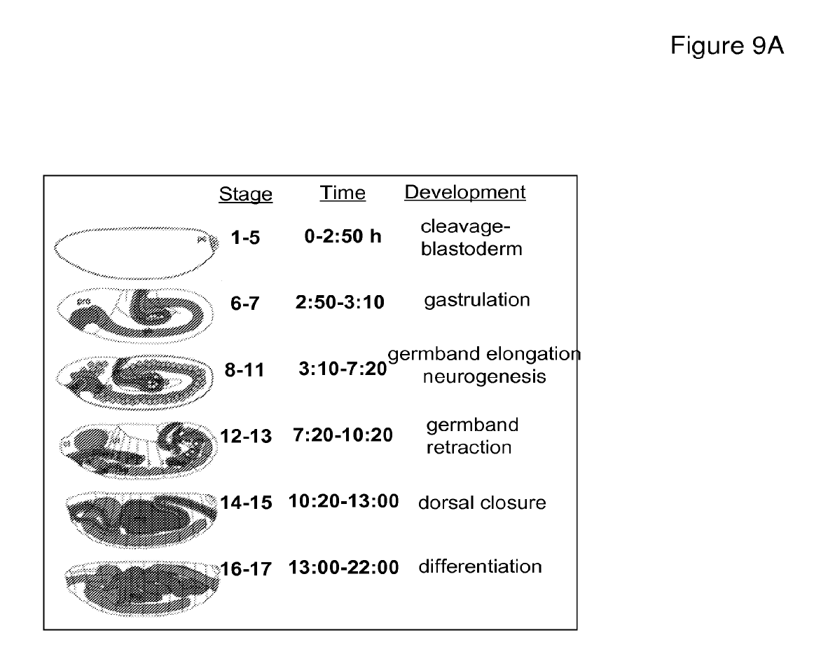

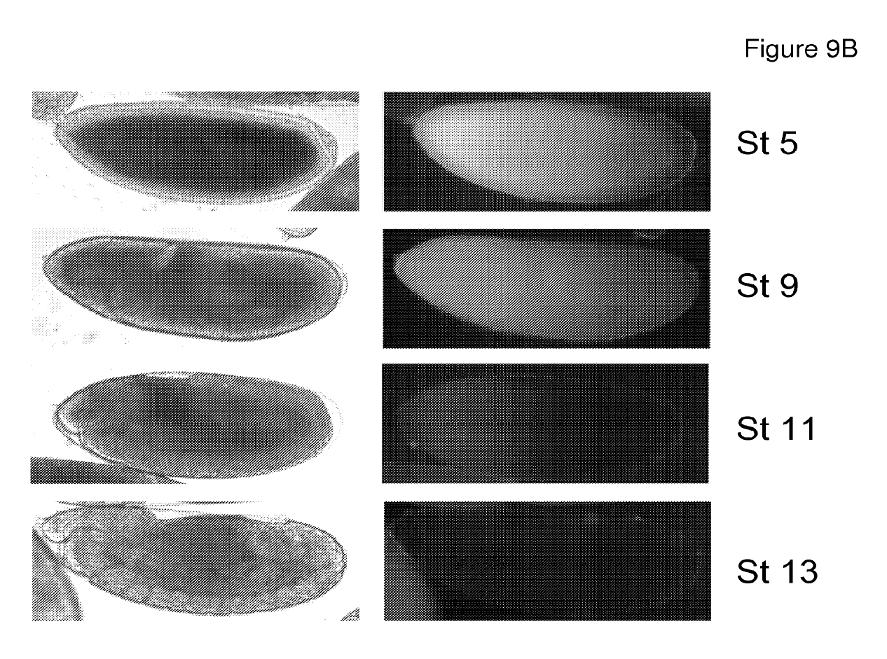

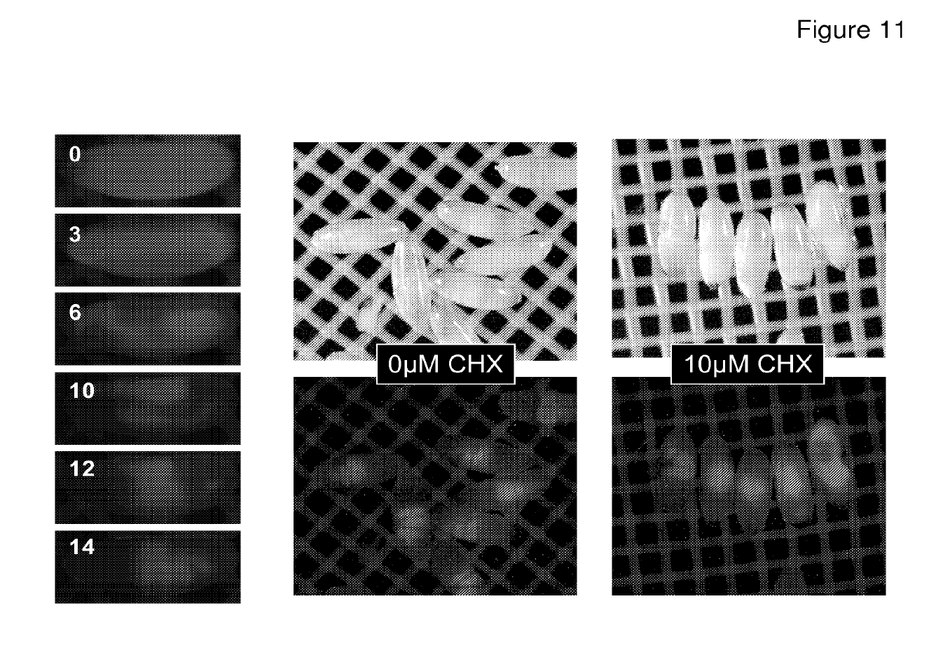

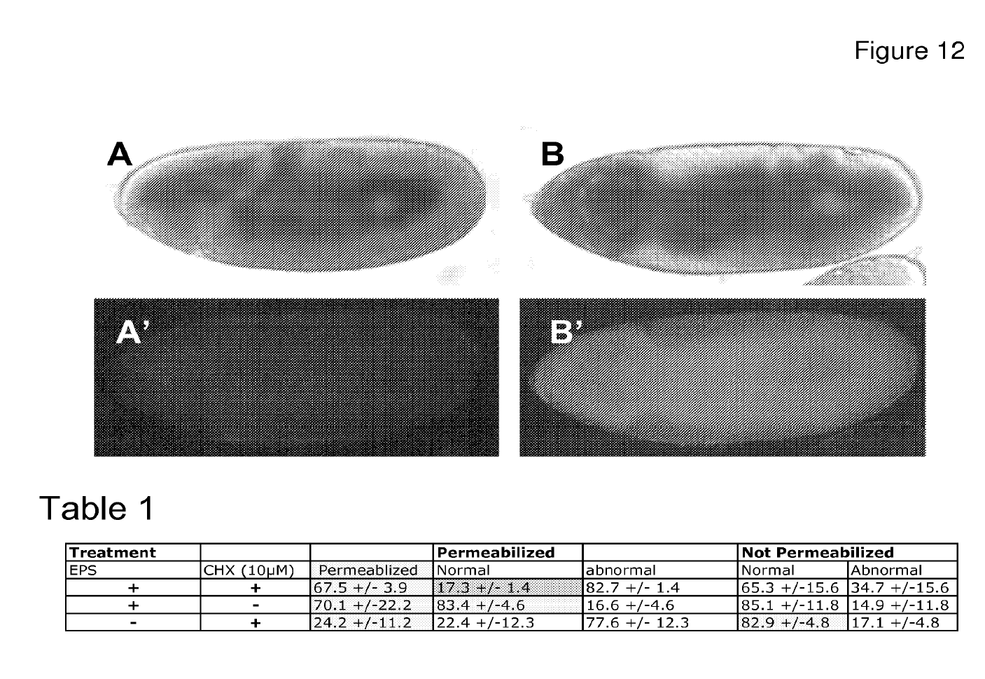

The embryo develops into a larva in 22-24 hours at 25° C. (refer to FIG. 9A). Several endpoints in this progression have proven highly effective to judge normal versus abnormal developmental processes in flies carrying genetic mutations or modifications. We investigated a variety of morphological events leading up to stage 17 for potentially easily identifiable endpoints. Through monitoring development under fluorescence time-lapse imaging we observed the highly dynamic pattern of blue fluorescence emitted from the auto-fluorescence of yolk proteins in the embryo. At preblastodem stages a relatively uniformly dispersed blue fluorescence is seen across the embryo (FIG. 11A). Between 3-6 hours of development this pattern changes drastically as the yolk proteins are pushed aside during germband elongation (refer to FIG. 9A). At ˜10 hours of development the germband retracts and anterior and posterior extensions of the presumptive midgut fuse to complete the intestinal tract by 12 hours (FIG. 11). Blue fluorescence is seen to consolidate in the lumen of the gut initially as one large mass in the newly fused midgut (FIG. 11A, 12 hrs). The midgut undergoes three circular constrictions at Stage 16 (FIG. 11A, 14 hrs). The compartmentalized strong blue fluorescence in the midgut makes normally developed Stage 16/17 embryos easily discernable under fluorescence (FIG. 11A, 14 hrs).

We next tested the utility of this endpoint to examine small molecule toxicity by exposing permeabilized embryos to cycloheximide (CHX), a potent protein synthesis inhibitor and teratogen. EPS-treated 3 hr old embryos were incubated in the presence (FIG. 11 D, E) or absence (FIG. 11A,B,C) of 10 μM CHX at 18° C. for 20 hours (equivalent to 10 hours at 25° C.) and examined under blue fluorescence. Control embryos consistently exhibited characteristic 3-4 compartments in the midgut due to constrictions at Stage 16 (FIG. 11A-C). In contrast, CHX treatment induced gut malformation as seen by the blue fluorescence centrally localized in a “ball” of tissue or dispersed anteriorly and posteriorly (FIG. 11D, E). These results indicate the pattern of blue fluorescence can be used as an easily distinguishable endpoint to evaluate small molecule toxicity in embryonic development. Similar assays can be performed using other visually distinguishable endpoints.

### Example 13

### Monitoring Teratogenic Effects Using a Patterning GFP Fusion Protein

We next sought to determine the feasibility of using an early embryogenesis endpoint as an indicator of teratogenic effects. In separate experiments using flies carrying the GFP reporter for the Dorsal protein we determined that CHX had profound effects on the morphogenetic movements of the embryo in the first 3-5 hours of development (see FIG. 13 below). During these stages formation of the cephalic furrow and germband elongation serve as landmarks that are easily identifiable under differential interference contrast (DIC) microscopy (see FIG. 8). We found that exposure of 2.5 hr old permeabilized embryos to 10 μM CHX for 1.5 hours results in disruption of cephalic furrow formation (compare FIG. 12A to 12B). We took advantage of this easily score-able CHX phenotype of disrupted cephalic furrow formation to assess the efficacy of EPS for permeabilization.

Treatment with EPS (1:10 diluted, 45″-1′15″ exposures) resulted in ˜70% of the embryos displaying permeabilization as determined by presence of rhodamine B fluorescence (Table 1). This is in contrast to only 24% showing dye uptake without EPS treatment (Table 1). With CHX (10 μM) treatment more than 80% of EPS treated embryos displayed an abnormal formation of the cephalic furrow. Without CHX treatment, roughly 17% of permeabilized embryos were abnormal (i.e. >80% were normal). The same degree of normal development was seen in non-permeabilized embryos. Furthermore, the same degree of normal development was observed in non-permeabilized embryos in the presence of CHX, indicating the EPS treatment is required for entry of CHX under these conditions. A somewhat lower, rate of normal development was seen in embryos that were not apparently permeablized in the preparation of EPS treatment and CHX exposure. This likely reflects an inability to determine the lowest levels of permeability via visual determination of rhodamine B red fluorescence. Together the data demonstrate that EPS treatment is an enabling step for delivery of CHX to the embryo.

### Example 14

### Genetic Markers as Readouts of Embryo Development

Our previous studies investigating methylmercury (MeHg) toxicity in Drosophila embryos demonstrated the effectiveness of green fluorescent protein (GFP) expression in the nervous system in transgenic flies as a reporter of abnormal neural development with MeHg exposure. We sought to examine earlier markers of development. In addition, some markers are coherent reporters of signaling pathway activity and offer the possibility to readout effects specific to the mechanism of that pathway.

We turned to the Dorsal-GFP transgenic fusion construct reporter to further examine the effects of the teratogen CHX. The Dorsal protein is the Drosophila NF-κB homolog which is central to signaling that establishes the dorsal-ventral axis in the embryo. Dorsal protein localizes to ventral cell nuclei where it acts to define the D/V axis. Delotto, et al. using a Dorsal-GFP fusion protein demonstrated dynamic shuttling of Dorsal in and out of the nucleus with each mitotic division in the preblastoderm embryo.

Using this same Dorsal-GFP construct we see dynamic distribution of Dorsal-GFP in and out of the ventral nuclei of an EPS treated pre-blastoderm embryos (FIG. 13A, B). Exposure to CHX causes gross deformities in the pattern of nuclear divisions and Dorsal distribution in preblastoderm embryos (FIG. 13C-E). In many cases these early embryos are seen to stall in development. Notably, Dorsal accumulation in nuclei occurs to high levels in very early embryos when the syncytial nuclei first appear at the embryo surface (FIG. 13C). Embryos affected at slightly later stages show irregular nuclear Dorsal accumulation (FIG. 13D) as well as varied sizes in the nuclei present (FIG. 13E). It is interesting to note that these deformities occur more frequently in the anterior portion of the embryo, consistent with the gradient of dye dispersal seen in EPS treated embryos.

### Example 15

### Engrailed GFP Studies

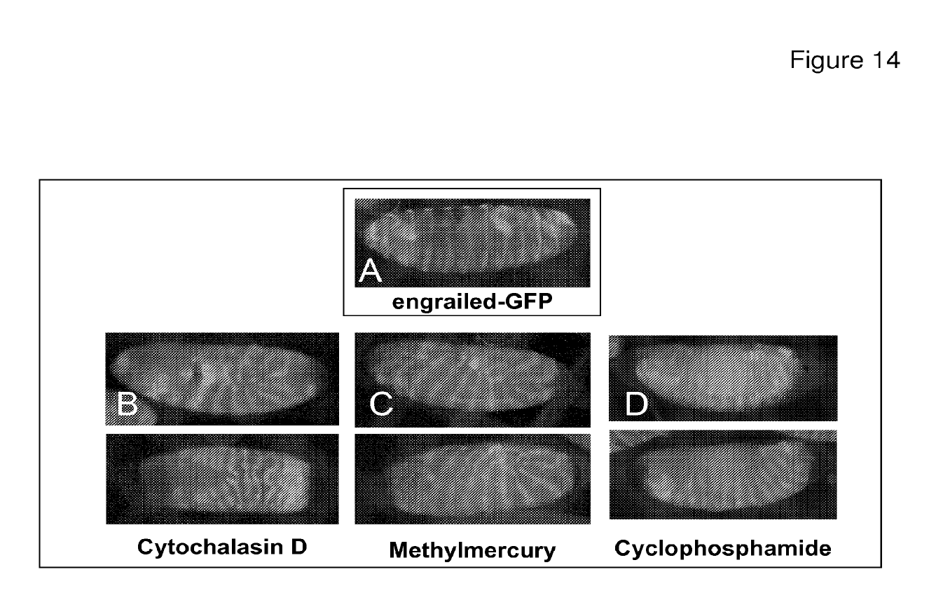

The engrailed gene encodes a homeodomain transcription factor that is highly conserved in all metazoans. Engrailed is a segment polarity gene whose expression marks the anterior boundary of each segment in the embryo. Engrailed also function in neural development to define developing brain regions and regulate growth of axons. Expression of GFP in embryonic engrailed cells in a late stage embryo can be seen in FIG. 14A.

Exposure of EPS-permeabilized embryos to a number of known teratogens results in grossly abnormal patterns of engrailed expression. Cytochalasin D is a potent inhibitor of actin polymerization and inhibitor of protein synthesis. Its teratogenic effects include exencephaly, hypognathia, and neural tube defects. We see that Cytochalasin D induces a stalling of embryo development at Stage 11 with gaps in the Engrailed band of cells in several segments (FIG. 14B). As well, a medial split in the engralied bands in the anterior ventral portion of the embryo is indicative of interruption of signaling events controlling ventral furrow formation and head formation (FIG. 14B, lower panel).

Methylmercury (MeHg) is an environmental toxin that preferentially targets embryonic neural development. We see that MeHg similarly stalls the embryo at Stage 11 and disrupts the regularity of engrailed banding pattern (FIG. 14C). This is consistent with the known activity of MeHg in inhibiting protein synthesis, as well as being an inhibitor of cell migration.

Cyclophosphamide has teratogenic effects giving rise to cleft palate and limb defects in mice and rats. We see that cyclophosphamide results in an overall shortening of the embryo and generates breaks in the bands of engrailed expressing cells (FIG. 14D). Thus, these GFP reporter demonstrates the potential for robust readout teratogenic activity.

### Example 16

### Detection of a Neural Developmental Endpoint after Treatment with EPS

The effectiveness of a neural specific fluorescent reporter in a transgenic strain of fly was assessed after treatment of the embryos with a working solution of EPS. The elav gene is a neural-specific gene that encodes an RNA binding protein homologous to the vertebrate Hu gene. Elav expression is restricted to neuronal precursors and neurons in a highly specific manner and has been used extensively as a neural marker in embryonic development by the Drosophila community Constructs with the elav promoter fused to the coding region of green fluorescent protein (GFP) show a faithful reproduction of expression pattern when compared to the elav protein antigen. Promoter specific expression can also be achieved with the heterologous yeast Gal4-UAS transactivation construct, a powerful and commonplace tool in fly methodology.

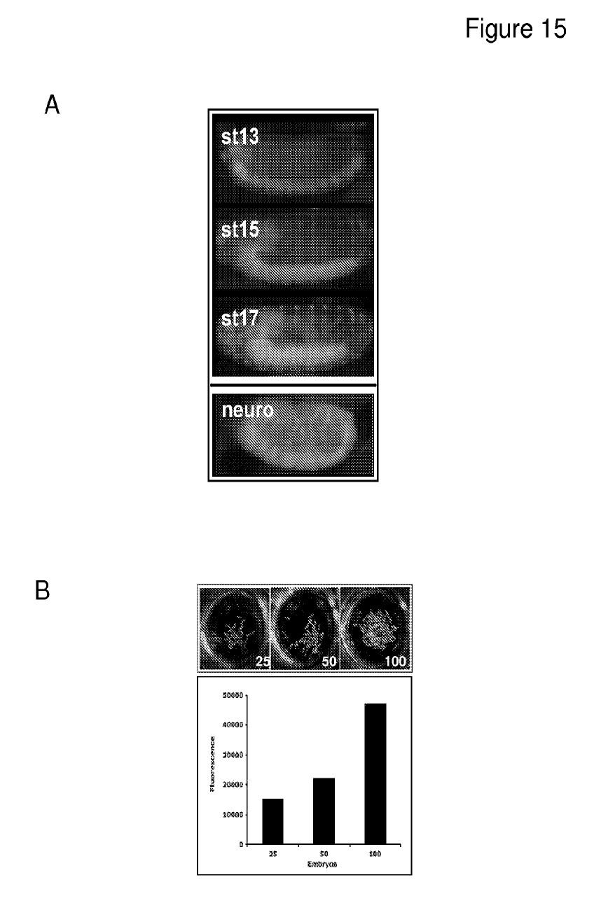

A strain carrying a recombinant X chromosome with the elav promoter fused to the Gal4 coding sequence and CD8-GFP coding sequence downstream of the UAS element (altogether denoted as EG4>GFP) was obtained. This stable line constitutively expresses a membrane-tethered GFP in neurons. Analysis of EG4>GFP embryos at various ages using standard epiflourescence microscopy revealed the stage specific developments of the nervous system (FIG. 15A). The intensity of the GFP signal was very robust, showing an increase with time of development as a result of the propagation of neuronal cells from neuroblast precursors as the embryo matures

In assays to optimize normal development after EPS (1:10 dilution) permeabilization, various compositions of the reservoir medium in development assays in Nitex® baskets were analyzed. It was found that Sang's M3 cell culture medium supplemented with bactopeptone-yeastolate extract (M3/BPYE, a standard composition for Drosophila cell culture) can result in as many as 30% of the EG4>GFP embryos hatching to larvae (data not shown). In this sample, more than 50% of the unhatched embryos displayed GFP fluorescence of significant intensity. As shown above, other media for Drosophila embryo culture are also useful for the culturing of embryos after treatment with EPS. Maturation of embryos to mobile organisms such as larva or adult flies is neither required, nor preferred for high throughput screening as automated imaging cannot be performed.

One outcome of altered neurogenesis is an overabundance of NBs, with the penultimate endpoint of an excess of neurons. Spontaneous neurogenic embryos typically occurs in a population of >500 embryos, and can be seen to display a broad expression of GFP across the embryo due to the overabundance of neurons specified (FIG. 15A). We predict this outcome would yield a significant quantitative increase in flourescence signal in EG4>GFP flies. We also predicted such an increase will be amenable to detection in a standard fluorescence plate reader.

As proof of principle, a 96-well plate was seeded with an increasing number of Stage 14-16 EG4>GFP embryos to determine the ability to discriminate an increase in fluorescent intensity from embryos presented in this orientation. As shown in FIG. 8, an increase in fluorescence intensity proportionate with embryo number could be detected by a standard FLOUstar Galaxy fluorescence plate reader (BMG Labtechnologies). Importantly, a robust signal was obtained with as few as 25 embryos in the bottom of the well (FIG. 15B). These data illustrate the feasibility of adapting a neurogenesis endpoint, or other fluorescently monitorable endpoints, to a straightforward fluorescence determination in a plate reader.

### Example 17

### Automated Instrumentation for a Drosophila Embryo Assay

It is desirable for high-throughput implementation of this embryo assay to provide automation with instrumentation for at least two steps: distribution of embryos to the multiwell format and endpoint fluorescent image capture and quantitation. Two instruments are currently commercially available that will execute these steps: the Union Biometrica COPAS SELECT® sorter (www.unionbio.com/products/overview.html) and the BD Biosciences Pathway High Content Bioimager (www.bdbiosciences.ca/bioimaging/cell_biology/pathway/435/), respectively.

The COPAS® instrument has the ability to sort embryos by fluorescence intensity and other physical characteristics, a feature that could eventually be implemented to select for identically staged embryos at the outset of the chemical probe assay. Importantly, the COPAS® instrument also has a robotic arm interface able to distribute sorted embryos to multiwell formats up to 384 wells.

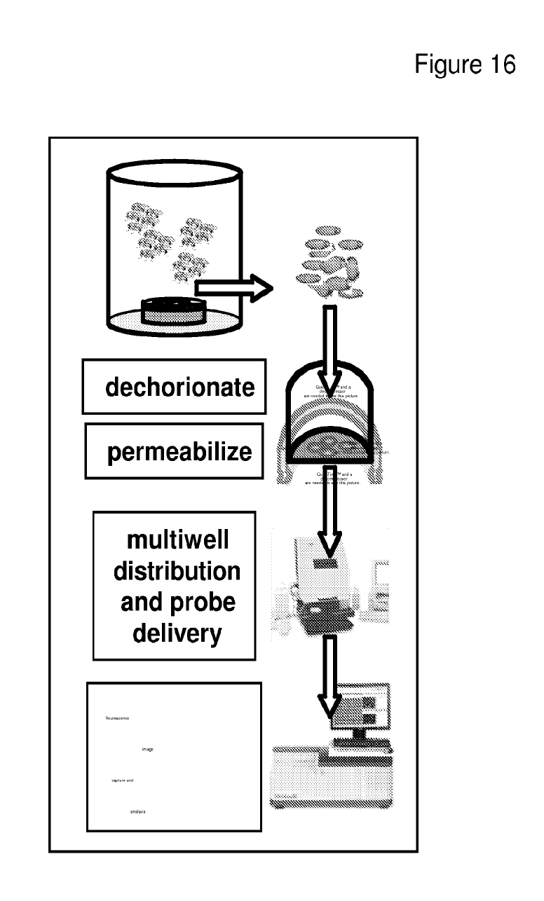

Ideally, endpoint fluorescence data can be obtained by straightforward measurements in a plate reader, as we demonstrate above. However, sample measurements may require higher resolution image acquisition for more specific endpoint analysis. For example, the fluorescent intensity increase may be best quantified by its relative distribution, e.g. GFP expression across the dimensions of the embryo (e.g., FIG. 16). The BD Pathway Bioimager is a spinning disc confocal system with automated capabilities of 3-D image capture from multiwell formats (up to 384 wells). The associated software has the ability to determine fluorescence intensity by a variety of multi-parameter assays, one of which can resolve the field of fluorescence distribution. While these latter endpoints are inherently more complex than detecting global fluorescence in the embryo, they speak to the potential to access the high-information content in an organism-based assay that cannot be obtained in a cell culture assay.

As these instruments are likely to be in use in HTS assay platforms at academic and commercial screening centers, we anticipate that adaptation of our fly embryo assay to these instruments will be straight forward for a facility that is versed in this instrumentation.

### REFERENCES

Each of the references, patents, and GenBank numbers cited in the application are hereby incorporated by reference as if they were cited individually.
S. Artavanis-Tsakonas, C. Delidakis, R. G. Fehon, The Notch locus and the cell biology of neuroblast segregation, Annu Rev Cell Biol 7 (1991) 427-52.
S. Artavanis-Tsakonas, M. A. Muskavitch, B. Yedvobnick, Molecular cloning of Notch, a locus affecting neurogenesis in Drosophila melanogaster, Proceedings of the National Academy of Sciences of the United States of America 80 (1983) 1977-81.
J. C. Aster, W. S. Pear, S. C. Blacklow, Notch signaling in leukemia, Annual review of pathology 3 (2008) 587-613.
C. Berger, S. Renner, K. Luer, G. M. Technau, The commonly used marker ELAV is transiently expressed in neuroblasts and glial cells in the Drosophila embryonic CNS, Dev Dyn 236 (2007) 3562-8.
T. Bossing, G. Udolph, C. Q. Doe, G. M. Technau, The embryonic central nervous system lineages of Drosophila melanogaster. I. Neuroblast lineages derived from the ventral half of the neuroectoderm, Developmental biology 179 (1996) 41-64.
A. H. Brand, A. S. Manoukian, N. Perrimon, Ectopic expression in Drosophila, Methods in cell biology 44 (1994) 635-54.
J. G. Corbin, N. Gaiano, S. L. Juliano, S. Poluch, E. Stancik, T. F. Haydar, Regulation of neural progenitor cell development in the nervous system, Journal of neurochemistry 106 (2008) 2272-87.
S. M. Dellatore, A. S. Garcia, W. M. Miller, Mimicking stem cell niches to increase stem cell expansion, Current opinion in biotechnology (2008).
C. Q. Doe, Molecular markers for identified neuroblasts and ganglion mother cells in the Drosophila central nervous system, Development (Cambridge, England) 116 (1992) 855-63.
Echalier, G. Drosophila Cells in Culture. Academic Press. 1997. Cap. 1, p. 58.
FDA, United States Code of the Federal Regulations, Title 21 Part 182.60.
V. Hartenstein, Atlas of Drosophila Development, Editoin Edition, Cold Spring Harbor Press, New York, 1993.
Hill D: Chemical removal of the chorion from Drosophila eggs. Drosophila Info Serv 1945, 19:62.
IPCS: Limonene: International Program on Chemical Safety-Concise International Chemical Assesment Documents (CICADs). In.: IPCS:INCHEM; 2009.
A. Joutel, C. Corpechot, A. Ducros, K. Vahedi, H. Chabriat, P. Mouton, S. Alamowitch, V. Domenga, M. Cecillion, E. Marechal and others, Notch3 mutations in cerebral autosomal dominant arteriopathy with subcortical infarcts and leukoencephalopathy (CADASIL), a mendelian condition causing stroke and vascular dementia, Ann N Y Acad Sci 826 (1997) 213-7.
B. Limbourg, M. Zalokar, Permeabilization of Drosophila eggs, Developmental biology 35 (1973) 382-7.
P. Limonene, www.directchem.com/607dlimbugki.html.
A. Louvi, S. Artavanis-Tsakonas, Notch signalling in vertebrate neural development, Nat Rev Neurosci 7 (2006) 93-102.
L. H. Margaritis, F. C. Kafatos, W. H. Petri, The eggshell of Drosophila melanogaster. I. Fine structure of the layers and regions of the wild-type eggshell, Journal of cell science 43 (1980) 1-35.
Mazur P, Cole K W, Mahowald A P: Critical factors affecting the permeabilization of Drosophila embryos by alkanes. Cryobiology 1992, 29(2):210-239.
National Research Council. Scientific Fronteirs in Developmental Toxicology and Risk Assesment. Nat'l. Academy Press. 2000.
NTP, TR-347: Toxicology and Carcinogenesis Studies of d-Limonene (CAS No. 5989-27-5) in F344/N Rats and B6C3F1 Mice (Gavage Studies), online access (1990).
Opdyke D L: Monographs on fragrance raw materials. Food and cosmetics toxicology 1975, 13 suppl:825-826.
V. Orgogozo, W. B. Grueber, FlyPNS, a database of the Drosophila embryonic and larval peripheral nervous system, BMC developmental biology 5 (2005) 4.
Qu P, Zhao J C, Shen T, Hidaka H: TiO2-assisted photodegradation of dyes: A study of two competitive primary processes in the degradation of RB in an aqueous TiO2 colloidal solution. JOURNAL OF MOLECULAR CATALYSIS A-CHEMICAL 1998, 129:257-268.
S. Robinow, K. White, Characterization and spatial distribution of the ELAV protein during Drosophila melanogaster development, Journal of neurobiology 22 (1991) 443-61.
G. Ruvkun, O. Hobert, The taxonomy of developmental control in Caenorhabditis elegans, Science (New York, N.Y. 282 (1998) 2033-41.
A. Schmid, A. Chiba, C. Q. Doe, Clonal analysis of Drosophila embryonic neuroblasts: neural cell types, axon projections and muscle targets, Development (Cambridge, England) 126 (1999) 4653-89.
Strecker T R, McGhee S, Shih S, Ham D: Permeabilization, staining and culture of living Drosophila embryos. Biotech Histochem 1994, 69(1):25-30.
M. Virot, V. Tomao, C. Ginies, F. Visinoni, F. Chemat, Green procedure with a green solvent for fats and oils' determination. Microwave-integrated Soxhlet using limonene followed by microwave Clevenger distillation, Journal of chromatography 1196-1197 (2008) 147-52.

1. A method of permeabilizing an insect embryo comprising a chorion layer and a waxy layer comprising sequentially:
removing a chorion layer from at least a portion of an insect embryo surface; and
contacting the surface of the embryo with the chorion layer removed with a working dilution of an embryo permeabilization solution (EPS) comprising a non-toxic, monocyclic terpene selected from the group consisting of limonene, carvone, and cineole and at least one non-toxic surfactant, whereby the insect embryo is permeabilized.

2. The method of claim 1, further comprising transferring the permeabilized embryo to growth media.

3. The method of claim 2, wherein at least a portion of the embryo surface is exposed for gas exchange.

4-6. (canceled)

7. The method of claim 1, wherein the embryo is permeable to a compound having a molecular weight of up to 1500 Da.

8-9. (canceled)

10. The method of claim 1, further comprising contacting the permeabilized embryo with a detectable label.

11. The method of claim 10, wherein contacting the permeabilized embryo with a detectable label is performed to detect permeabilization or viability of the embryo.

12-16. (canceled)

17. The method of claim 1, further comprising contacting the embryo with an agent suspected of comprising a biological activity.

18. The method of claim 17, wherein the biological activity comprises toxin activity.

19. The method of claim 17, further comprising observing the embryo contacted with the agent as compared to an embryo not contacted with the agent, wherein a change in the embryo contacted with the agent indicates that the agent comprises biological activity.

20. The method of claim 17, wherein the agent results in an alteration of transcription from a promoter.

21. The method of claim 20, wherein the alteration in transcription from a promoter is detected using a reporter gene.

22-28. (canceled)

30. The method of claim 1, wherein the surfactant is selected from the group consisting of alkyl poly(ethylene oxide), ethoxylated coconut alcohol, alcohol ethoxylate (C₉₋₁₁), copolymers of poly(ethylene oxide) and poly(propylene oxide); alkyl polyglucosides; fatty alcohols; cocamide MEA, cocamide DEA; polysorbates, ethoxylated fatty alcohol ethers and lauryl ethers, ethoxylated alkyl phenols, octylphenoxy polyethoxy ethanol compounds, modified oxyethylated and/or oxypropylated straight-chain alcohols, polyethylene glycol monooleate compounds, polysorbate compounds, and phenolic fatty alcohol ethers.

31. The method of claim 1, wherein the surface of the embryo with the chorion layer removed is contacted with a working dilution of an embryo permeabilization solution (EPS) in an embryo incubation device comprising:
a sidewall enclosing a space comprising a top opening and a bottom opening;
a mesh bottom covering the bottom opening; and
a base for elevating the mesh bottom comprising at least two openings,
wherein the embryo incubation device is placed in a chamber containing a working solution of EPS such that the EPS contacts the mesh bottom of the device.

32-35. (canceled)

36. An embryo incubation device comprising:
a sidewall enclosing a space comprising a top opening and a bottom opening;
a mesh bottom covering the bottom opening; and
a base for elevating the mesh bottom comprising at least two openings.

37. The embryo incubation device of claim 36, wherein the embryo incubation device is attached to at least one other embryo incubation device.

38. An embryo permeabilization solution for use in the method of claim 1.

39. A kit comprising an embryo permeabilization solution for use in the method of claim 1.
